(19) United States
(12) Patent Application Publication
(10) Pub. No.: US 2026/0184738 A1
(43) Pub. Date: Jul. 2, 2026

(54) EXTENDED PURINE TRICYCLIC AND BICYCLIC NUCLEOSIDES AND NUCLEOTIDES FOR USE AS ANTIVIRAL THERAPEUTICS

(71) Applicant: UNIVERSITY OF MARYLAND, BALTIMORE COUNTY, Baltimore, MD (US)

(72) Inventor: Katherine L. RADTKE, Ellicott City, MD (US)

(21) Appl. No.: 19/549,290

(22) Filed: Feb. 25, 2026

Related U.S. Application Data
(63) Continuation of application No. PCT/US2024/044178, filed on Aug. 28, 2024.
(60) Provisional application No. 63/579,432, filed on Aug. 29, 2023.

(51) Int. Cl. C07H 19/24; A61K 31/7056; A61K 31/7068; A61K 31/7072; A61P 31/14

(52) U.S. Cl. CPC C07H 19/24; A61K 31/7056; A61K 31/7068; A61K 31/7072; A61P 31/14

(57) ABSTRACT

Compounds, methods, and compositions for treating or preventing viral infections using nucleoside compounds. The nucleoside compounds have increased antiviral activity potential with the result of inhibiting at least one of flaviviruses, herpesviruses, polyomaviruses, alphaviruses, enteroviruses, filoviruses matonaviruses, phenuiviruses, Hepatitis B virus, and/or coronaviruses.

### CROSS REFERENCE TO RELATED APPLICATIONS

This application is a continuation application claiming priority to International Patent Application No. PCT/US2024/044178 filed on 28 Aug. 2024, which claims priority to U.S. Provisional Patent Application No. 63/579,432 filed on Aug. 29, 2023 in the name of Katherine L. Radtke and entitled “Extended Purine Tricyclic and Bicyclic Nucleosides and Nucleotides for Use as Antiviral Therapeutics,” both of which are hereby incorporated by reference herein in their entirety.

### FIELD

The present invention is directed to nucleoside analogue compounds, compositions comprising same, and methods for treating or preventing any one of coronaviruses, herpesviruses, alphaviruses, enteroviruses, polyomaviruses, filoviruses, matonaviruses, phenuiviruses, Hepatitis B virus, and/or flaviviruses using said nucleosides analogues. Specifically, the present invention provides for the design and synthesis of extended and expanded nucleoside and nucleotide scaffolds having increased antiviral activity potential that can inhibit several viruses.

### BACKGROUND

Viruses are small infectious agents that can only multiply within the cells of animals, plants, and bacteria. The structures of viruses are simple compared to living cells and contain a small haploid DNA or RNA genome and a protein or glycoprotein coat called a capsid. In addition, some viruses called enveloped viruses are surrounded by a lipid membrane.

A number of viruses appear on the United States National Institutes of Allergy and Infectious Disease (NIAID) list of Emerging Diseases/Pathogens list, which include Coronaviruses (e.g., SARS-1, SARS-2, Middle East respiratory syndrome (MERS), COVID-19, and mutants thereof), Flaviviruses (e.g., Dengue, Zika, yellow fever, tickborne encephalitis, HCV, and West Nile), Herpesviruses (e.g., herpes simplex virus 1, herpes simplex virus 2, varicella-zoster virus, Epstein-Barr virus, cytomegalovirus, Human herpesvirus-6, Human herpesvirus-7, and Kaposi's sarcoma herpes virus), alphavirus (e.g., eastern equine encephalomyelitis (EEE), Venezuelan equine encephalomyelitis (VEE) and western equine encephalomyelitis (WEE), Chikungunya), enteroviruses (e.g., echovirus and coxsackievirus), Filoviruses (e.g., Ebola virus, Sudan virus, and Marburg virus), matonaviruses (e.g., Rubella, Rustrela, and Ruhugu), and phenuiviruses (e.g., Rift Valley Fever virus) to name a few.

Filoviruses are enveloped viruses with a genome consisting of one linear single-stranded RNA segment of negative polarity. The viral genome encodes 7 proteins. Nucleoprotein (NP), virion protein 35 kDa (VP35) and virion protein 30 kDa (VP30) are associated with the viral ribonucleoprotein complex. Members of the filovirus genus include Zaire Ebola virus, Sudan Ebola virus, Reston Ebola virus, Cote d'Ivoire Ebola virus and Marburg virus. Ebola and Marburg viruses can cause severe hemorrhagic fever and have a high mortality rate. Ebola virus (Zaire and Sudan species) was first described in 1976 after outbreaks of a febrile, rapidly fatal hemorrhagic illness were reported along the Ebola River in Zaire (now the Democratic Republic of the Congo) and Sudan. The natural host for Ebola viruses is still unknown but is widely speculated to be bats. Marburg virus, named after the German town where it was first reported in 1967, is primarily found in equatorial Africa. The host range of Marburg virus includes non-human and human primates.

Viruses in the genus flavivirus are known to cause viral hemorrhagic fevers (VHFs). Flaviviruses are enveloped viruses with a genome consisting of one linear single-stranded RNA segment of positive polarity. The polyprotein is co- and post-transcriptionally cleaved by cell signal peptidase and the viral protease to generate individual viral proteins. Viral structural proteins include capsid (C), precursor to M (prM), minor envelope (M) and major envelope (E). Members of the flavivirus genus include yellow fever virus, Apoi virus, Aroa virus, Bagaza virus, Banzi virus, Bouboui virus, Bukalasa bat virus, Cacipacore virus, Carey Island virus, Cowbone Ridge virus, Dakar bat virus, dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Phenh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, tick-borne encephalitis virus, Tyuleniy virus, Uganda S virus, Usutu virus, Wesselsbron virus, West Nile virus, Yaounde virus, Yokose virus, Zika virus, cell fusing agent virus and Tamana bat virus.

Coronaviruses are enveloped viruses, having a capsid exhibiting a helical symmetry. They have a single-stranded positive sense RNA genome and are capable of infecting cells from birds and mammals. The viruses, which are members of this very wide family, are known to be causative agents for the common cold (for example, human coronaviruses (HCoV) 229E and OC43), bronchiolitis (for example NL63 virus) or even some forms of pneumoniae, e.g., as those observed during the SARS (such as the Severe Acute Respiratory Syndrome Coronavirus, SARS-CoV) epidemic. In January 2020 the World Health Organization reported infection from a new novel coronavirus SARS-CoV-2 (COVID-19 disease) which has shown a higher mortality rate than MERS-CoV or SARS-CoV and exhibits an ongoing risk of human-to-human transmission.

Viral hepatitis such as hepatitis B is an important and major disease threatening the life and health of people, and the fundamental approach for treatment of hepatitis B is an antiviral treatment. Although there exists a highly effective HBV vaccine, it is not readily available in some nations (e.g., developing nations). At present, clinically effective drugs against hepatitis B virus are mainly interferons or L-nucleosides (such as Lamivudine). However, the effective rate of interferon treatment is just 30-50% and has dose-dependent toxicity and side-effects. Lamivudine has pronounced activity against hepatitis B virus but may result in drug resistance during long-term administration, which is likely since treatment for chronic HBV is life-long. With the increased interest toward effective treatment of HBV, other L-nucleosides were developed such as Emtricitabine, telbivudine (L-dT), 1-(2-deoxy-2-fluoro-β-L-arabinofuranosyl)-5-methyluracil (Clevudine, L-FMAU), and Elvucitabine, or L-d4FC.

There are relatively few prophylactic or therapeutic agents for treatment of viral diseases caused by Coronaviruses, Alphaviruses, Enteroviruses, Herpesviruses. Flaviviruses, Matonaviridae, Phenuiviridae, Hepatitis B virus, and/or Filoviruses. The need for new and more effective antiviral therapeutics, particularly those targeting emerging and reemerging infectious diseases and pathogens continues to increase. Thus, in light of the above discussion, there is a need for discovering and providing new and more efficient antiviral drugs.

### SUMMARY

The present invention provides for the design and synthesis of extended and expanded nucleoside and nucleotide scaffolds with the ability to retain their potency against viral diseases caused by a virus including, but not limited to, Coronaviruses, Polyomaviruses, Herpesviruses, Alphaviruses, Enteroviruses, Flaviviruses, Matonaviruses, Phenuiviruses, Hepatitis B virus, and Filoviruses. The compounds described herein are obtained by inserting a five-membered ring in between the two components of the purine ring system, thereby expanding and extending the heterocyclic base.

In some aspects, a nucleoside compound is described selected from at least one of formula (I) or formula (II):

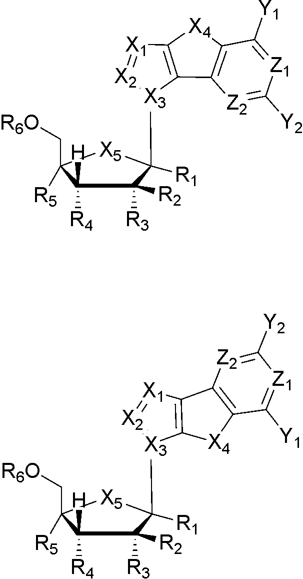

wherein
X₁ and X₂ are each independently selected from C, O, N, or S;
X₃ is C or N;
X₄ is O, NH, S, or CH₂;
X₅ is O, NH, S, or CH₂;
Y₁ and Y₂ are each independently selected from H, OH, SH, ═O, F, NH₂, NH-alkyl, O-alkyl, NH—OH, alkyl, CF₃, Cl, CN, or N₃;
Z₁ and Z₂ are each independently selected from C or a heteroatom;
R₁ is H or cyano;
R₂ and R₃ are each independently selected from H, OH, F, Cl, alkyl, ethynyl, or various prodrugs (e.g., amino acid prodrugs);
R₄ is H, OH, N₃, or various prodrugs (e.g., amino acid prodrugs);
R₅ is H, F, ethynyl or cyano; and
R₆ is H, monophosphate, diphosphate, triphosphate, or various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof;
   and/or formula (III)

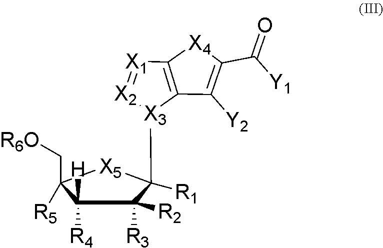

wherein
X₁ and X₂ are each independently selected from C, O, N, or S;
X₃ is C or N;
X₄ is O, NH, S, or CH₂;
X₅ is O, NH, S, or CH₂;
Y₁ is OH, NH₂, NH-alkyl, O-alkyl or NH—Ar
Y₂ is NH₂, NH-alkyl, O-alkyl or NH—Ar
R₁ is H or cyano;
R₂ and R₃ are each independently selected from H, OH, F, Cl, alkyl, ethynyl, or various prodrugs (e.g., amino acid prodrugs);
R₄ is H, OH, N₃, or various prodrugs (e.g., amino acid prodrugs);
R₅ is H, F, ethynyl or cyano; and
R₆ is H, monophosphate, diphosphate, triphosphate, or various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof,
   or a pharmaceutically acceptable salt, isomer, hydrate, prodrug or solvate thereof.

In another aspect, a pharmaceutical composition is described comprising at least one nucleoside compound and at least one pharmaceutically acceptable carrier, wherein the at least one nucleoside compound is selected from at least one of formula (I) or formula (II):

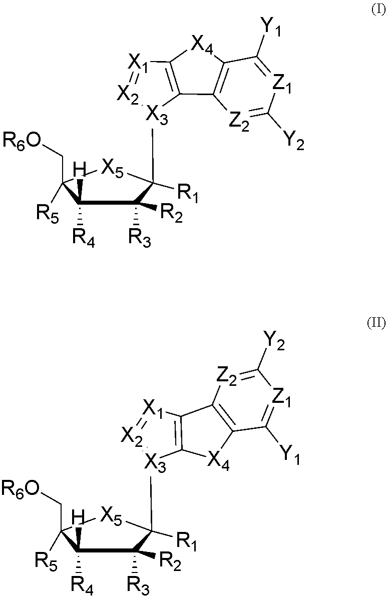

wherein
X₁ and X₂ are each independently selected from C, O, N, or S;
X₃ is C or N;
X₄ is O, NH, S, or CH₂;
X₅ is O, NH, S, or CH₂;
Y₁ and Y₂ are each independently selected from H, OH, SH, ═O, F, NH₂, NH-alkyl, O-alkyl, NH—OH, alkyl, CF₃, Cl, CN, or N₃;
Z₁ and Z₂ are each independently selected from C or a heteroatom;
R₁ is H or cyano;
R₂ and R₃ are each independently selected from H, OH, F, Cl, alkyl, ethynyl, or various prodrugs (e.g., amino acid prodrugs);
R₄ is H, OH, N₃, or various prodrugs (e.g., amino acid prodrugs);
R₅ is H, F, ethynyl or cyano; and
R₆ is H, monophosphate, diphosphate, triphosphate, or various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof;
   and/or formula (III)

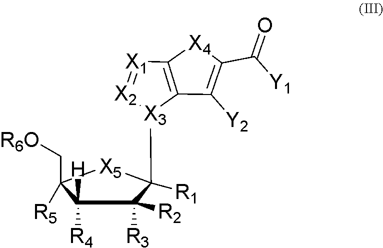

wherein
X₁ and X₂ are each independently selected from C, O, N, or S;
X₃ is C or N;
X₄ is O, NH, S, or CH₂;
X₅ is O, NH, S, or CH₂;
Y₁ is OH, NH₂, NH-alkyl, O-alkyl or NH—Ar
Y₂ is NH₂, NH-alkyl, O-alkyl or NH—Ar
R₁ is H or cyano;
R₂ and R₃ are each independently selected from H, OH, F, Cl, alkyl, ethynyl, or various prodrugs (e.g., amino acid prodrugs);
R₄ is H, OH, N₃, or various prodrugs (e.g., amino acid prodrugs);
R₅ is H, F, ethynyl or cyano; and
R₆ is H, monophosphate, diphosphate, triphosphate, or various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof,
   or a pharmaceutically acceptable salt, isomer, hydrate, prodrug or solvate thereof.

In still another aspect, a method for treating and/or preventing a viral infection in a subject is described, wherein the viral infection is caused by at least one of a coronavirus, a herpesvirus, an alphavirus, a polyomavirus, an enterovirus, a filovirus, a matonavirus, a phenuivirus, Hepatitis B virus, and/or a flavivirus, comprising administration, to the subject, of a therapeutically effective amount of at least one nucleoside compound described herein, or a pharmaceutical composition described herein.

Other aspects, features and embodiments of the invention will be more fully apparent from the ensuing disclosure and appended claims.

### DETAILED DESCRIPTION

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As defined herein, “alkyl” group corresponds to a C₁-C₆ straight or branched-chain group, having the general formula of CₙH₂ₙ₊₁ including, but not limited to, methyl, ethyl, n-propyl, isopropyl, n-butyl, sec-butyl, isobutyl, t-butyl, n-pentyl, 1-methylbutyl, 2-methylbutyl, 3-methylbutyl, 1-ethylpropyl, 1,1-dimethylpropyl, 1,2-dimethylpropyl, 2,2-dimethylpropyl, n-hexyl, 4-methylpentyl, 3-methylpentyl, 2-methylpentyl, 1-methylpentyl, 1,3-dimethylbutyl, 1,2-dimethylbutyl, 3,3-dimethylbutyl, 1-methyl-2,2-dimethylpropyl, 1-ethylbutyl, 1-ethyl-2-methylpropyl, and 2-ethylbutyl. In addition, an alkyl group includes a C₃-C₆ cycloalkyl group having the general formula of CₙH₂ₙ₋₁ including, but not limited to, cyclopropyl, cyclobutyl, cyclopentyl, cyclopentenyl, and cyclohexyl.

As defined herein, a “halide” corresponds to fluoride, chloride, bromide or iodide.

As defined herein, an “aryl” group corresponds to a functional group derived from an aromatic ring (e.g., an aromatic hydrocarbon ring) by the removal of one hydrogen atom.

As defined herein, a “heteroatom” includes nitrogen, oxygen, sulfur, and silicon. In some embodiments, the heteroatom is nitrogen.

“Subject” as used herein refers to any vertebrate such as mammals, birds, reptiles, amphibians and fish including, but not limited to, a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, monkey, chimpanzee, and humans. In some embodiments, the subject is a human.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

As defined herein, “lipid phosphates” include, but are not limited to,

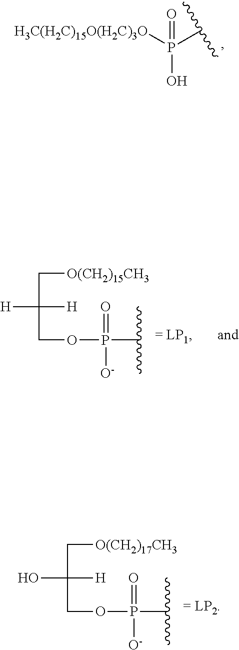

[symbol]

erein, “McGuigan ProTides” correspond to prodrugs of monophosphate nucleotide [symbol] hosphoramidate prodrugs (known as “ProTide” prodrugs). These nucleotide ProTide prodrugs were developed by Prof. McGuigan, for example as described in PCT Applications WO90/05736; WO90/10012; WO9629336; WO2000/047591; WO2001/083501; WO2001085749; WO2003/061670; and WO2005/012327, which are incorporated herein by reference in their entirety.

The McGuigan ProTides have been successfully applied to a vast number of nucleoside analogues with antiviral and anticancer activity. ProTides consist of a 5′-nucleoside monophosphate in which the two hydroxyl groups are masked with an amino acid ester and an aryloxy component which once in the cell is enzymatically metabolized to deliver free 5′-monophosphate, which is further transformed to the active 5′-triphosphate form of the nucleoside analogue. In some embodiments, the McGuigan Protide has the structure:

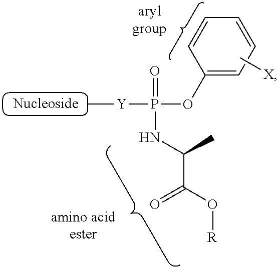

wherein
X is a substituted or unsubstituted aryl, naphthalene or biphenyl, wherein the substitutions include, but are not limited to, OH, NH₂, F, alkyl, O-alkyl, and/or NH-alkyl;
Z is the side chain of the amino acid including, but not limited to, methyl (i.e., the amino acid is alanine), and isopropyl (i.e., the amino acid is valine);
Y is O or CH₂; and
R is an alkyl or aryl group including, but not limited to, methyl, isopropyl, isobutyl, sec-butyl, or ethyl butyl.

### Tricyclic Compounds

The nucleoside compounds described herein are obtained by inserting a five-membered ring in between the two components of the purine ring system, thereby expanding and extending the heterocyclic base.

It should be appreciated that any of the nucleoside compounds described herein can be present as tautomers, or interchangeable forms, of the specific molecule described. Tautomers contemplated include the interchange between an enol form and a keto form, between a lactam form and a lactim form, between an amide form and an imidic acid form, and between an amine form and an imine form.

In a first aspect, the present invention broadly relates to a nucleoside compound selected from at least one of formula (I) or formula (II):

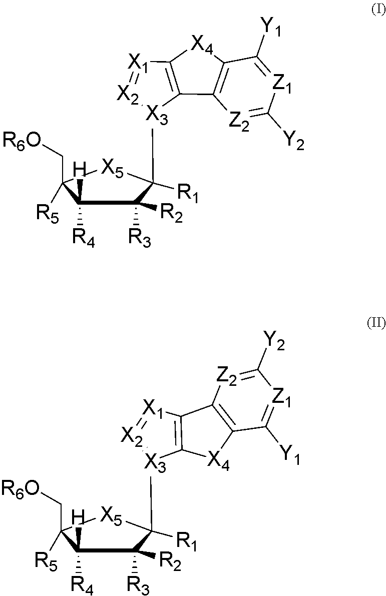

wherein
X₁ and X₂ are each independently selected from C, O, N, or S;
X₃ is C or N;
X₄ is O, NH, S, or CH₂;
X₅ is O, NH, S, or CH₂;
Y₁ and Y₂ are each independently selected from H, OH, SH, ═O, F, NH₂, NH-alkyl, O-alkyl, NH—OH, alkyl, CF₃, Cl, CN, or N₃;
Z₁ and Z₂ are each independently selected from C or a heteroatom;
R₁ is H or cyano;
R₂ and R₃ are each independently selected from H, OH, F, Cl, alkyl, ethynyl, or various prodrugs (e.g., esters, McGuigan ProTides, lipid phosphates, lipid esters, HepDirect, amino acid prodrugs);
R₄ is H, OH, N₃, or various prodrugs (e.g., esters, McGuigan ProTides, lipid phosphates, lipid esters, HepDirect, amino acid prodrugs);
R₅ is H, F, ethynyl or cyano; and
R₆ is H, monophosphate, diphosphate, triphosphate, or various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof;
   and/or at least formula (III):

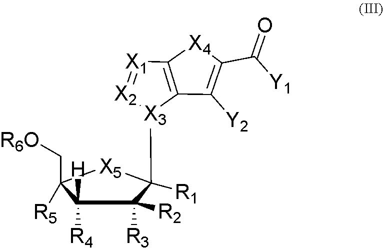

wherein
X₁ and X₂ are each independently selected from C, O, N, or S;
X₃ is C or N;
X₄ is O, NH, S, or CH₂;
X₅ is O, NH, S, or CH₂;
Y₁ is OH, NH₂, NH-alkyl, O-alkyl or NH—Ar
Y₂ is NH₂, NH-alkyl, O-alkyl or NH—Ar
R₁ is H or cyano;
R₂ and R₃ are each independently selected from H, OH, F, Cl, alkyl, ethynyl, or various prodrugs (e.g., esters, McGuigan ProTides, lipid phosphates, lipid esters, HepDirect, amino acid prodrugs);
R₄ is H, OH, N₃, or various prodrugs (e.g., esters, McGuigan ProTides, lipid phosphates, lipid esters, HepDirect, amino acid prodrugs);
R₅ is H, F, ethynyl or cyano; and
R₆ is H, monophosphate, diphosphate, triphosphate, or various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof,
   or a pharmaceutically acceptable salt, isomer, hydrate, prodrug or solvate thereof.

In some embodiments of the first aspect, the nucleoside compound is one of either formula (I) or (II), or a pharmaceutically acceptable salt, isomer, hydrate, prodrug or solvate thereof. In some embodiments, X₁═X₃═N and X₂ is C. In some embodiments, Z₁═Z₂═N. In some embodiments, X₄ is S. In some embodiments, X₁═X₃═N, X₂ is C, and Z₁═Z₂═N. In some embodiments, X₁═X₃═N, X₂ is C, and X₄ is S. In some embodiments, X₁═X₃═N, X₂ is C, X₄ is S, and Z₁═Z₂═N. In some embodiments, R₁═R₅═H. In some embodiments, R₂ and R₃ are the same as or different from one another and are selected from H, OH, F, or methyl. In some embodiments, R is OH or a prodrug. In some embodiments, R₆ is one of H, McGuigan ProTide, or triphosphate. In some embodiments, Y₁ and Y₂ are the same as or different from one another and are selected from H, OMe, NH₂, or ═O.

In some embodiments of the first aspect, the nucleoside compound is formula (III), or a pharmaceutically acceptable salt, isomer, hydrate, prodrug or solvate thereof. In some embodiments, X₁═X₃═N and X₂ is C. In some embodiments, Z₁═Z₂═N. In some embodiments, X₄ is S. In some embodiments, X₁═X₃═N, X₂ is C, and Z₁═Z₂═N. In some embodiments, X₁═X₃═N, X₂ is C, and X₄ is S. In some embodiments, X₁═X₃═N, X₂ is C, X₄ is S, and Z₁═Z₂═N. In some embodiments, R₁═R₅═H. In some embodiments, R₂ and R₃ are the same as or different from one another and are selected from H, OH, F, or methyl. In some embodiments, R is OH or a prodrug. In some embodiments, R₆ is one of H, McGuigan ProTide, or triphosphate. In some embodiments, Y₁ and Y₂ are the same as or different from one another and are selected from H, OMe, or NH₂.

In some embodiments of the first aspect, the nucleoside compound comprises at least one of:
CEM-001: 1-[5-(Pyrimidin-6-yl-4-one)imidazol-1-yl]-1-β-D-ribofuranose 2,3,5-Triol (compound 11)

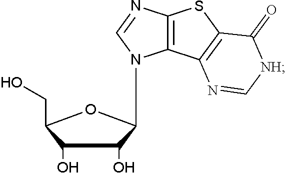

CEM-002: 1-[(5-Hydroxylimidazo[4′,5′:4,5]thieno[3,2-d]pyrimidin-3-yl-7-one)]-β-D-ribofuranose 2,3,5-Triol (compound 16)

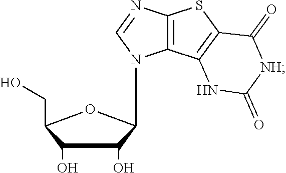

CEM-004: 1-[(5-carboxamide)-thieno[2,3-d]imidazol-3-yl]-β-D-ribofuranose 2,3,5-Triol (compound 6)

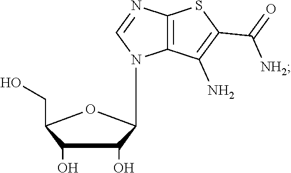

CEM-007: 1-[(5,7-diaminoimidazo-[4′,5′:4,5]thieno[3,2-d]pyrimidin-3-yl)]-β-D-ribofuranose-2,3,5-Triol (compound 21)

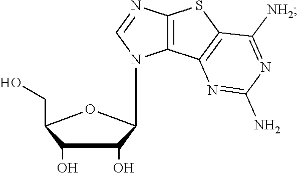

CEM-009: Nucleoside prodrug (compound 19)

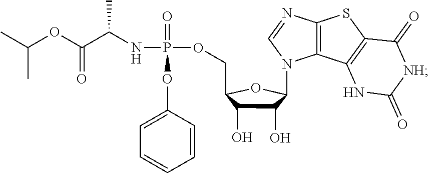

CEM-010: Nucleoside prodrug (compound 14)

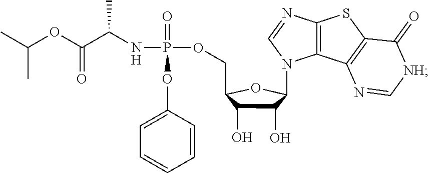

CEM-011: Nucleoside prodrug (compound 24a)

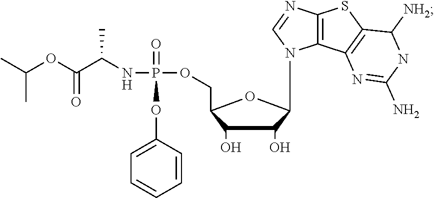

CEM-012: Nucleoside prodrug (compound 29)

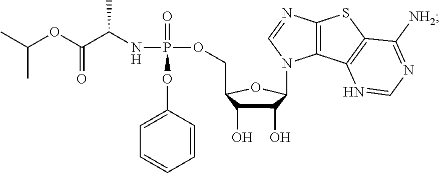

CEM-015: Nucleoside prodrug (compound 9)

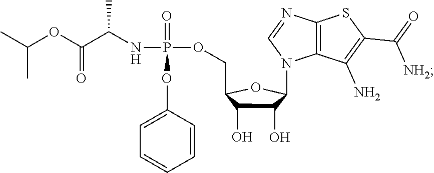

CEM-021: 1-[(5-Aminoimidazo[4′,5′:4,5]thieno[3,2-d]pyrimidin-3-yl-7-one)]-β-D-ribofuranose 2,3,5-Triol

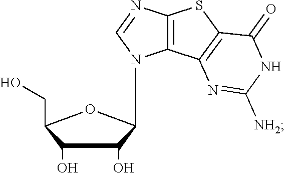

CEM-024: 2′-deoxy-2′-fluoro-2′-C-methyl-1′-[(5,7-diaminoimidazo-[4′,5′:4,5]thieno[3,2-d]pyrimidin-3-yl)]-β-D-ribofuranose (compound 34)

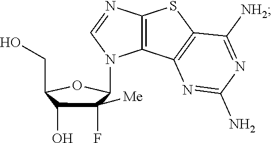

CEM-028: 1-[(5,7-dimethoxyimidazo-[4′,5′:4,5]thieno[3,2-d]pyrimidin-3-yl)]-β-D-ribofuranose-2,3,5-Triol

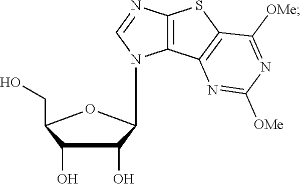

CEM-033: Nucleoside prodrug

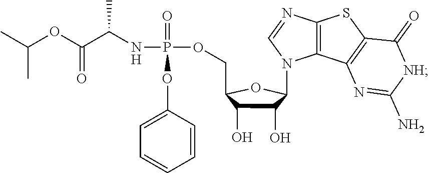

CEM-036: Nucleoside prodrug

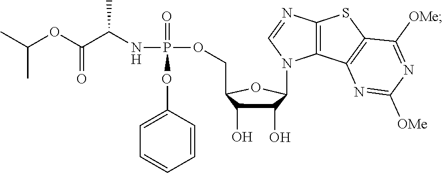

CEM-038: Trisphosphate compound (compound 38)

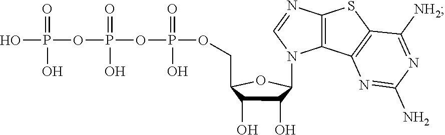

CEM-041: Nucleoside prodrug (compound 35a)

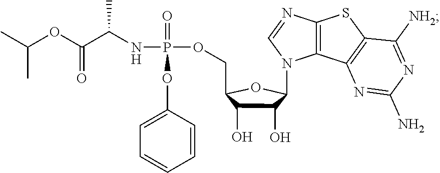

CEM-042: Nucleoside prodrug (compound 36a)

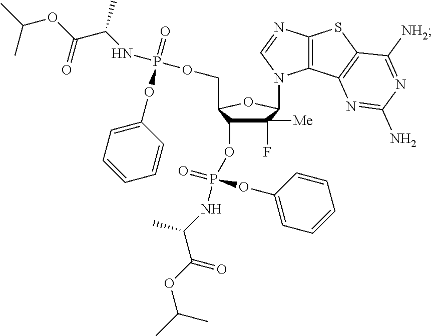

CEM-051: Trisphosphate compound (compound 39)

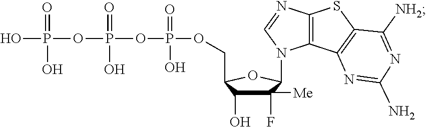

CEM-052: Nucleoside prodrug (compound 35b)

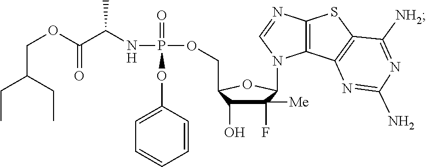

CEM-053: Nucleoside prodrug (compound 36b)

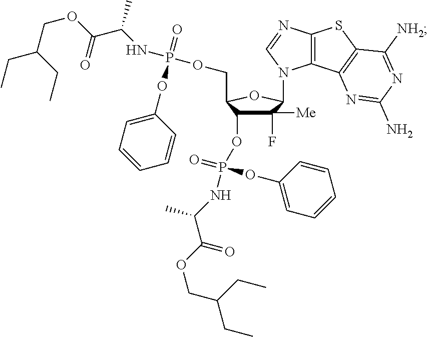

CEM-054: Nucleoside prodrug (compound 37a)

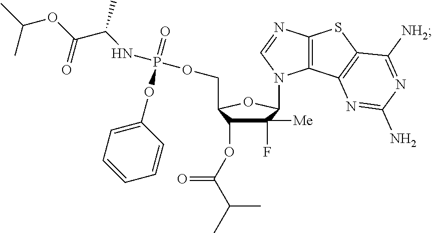

CEM-055: Nucleoside prodrug (compound 37b)

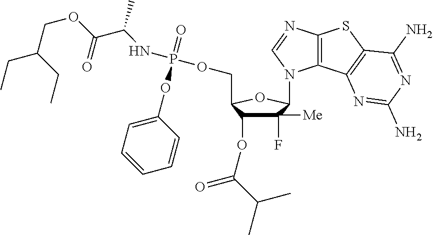

CEM-056: Nucleoside prodrug (compound 24b)

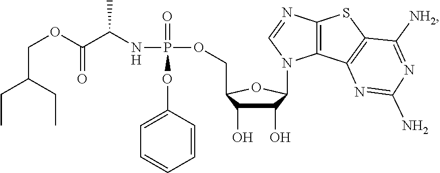

or a pharmaceutically acceptable salt, isomer, hydrate, prodrug or solvate thereof.

In some embodiments, the prodrug is an ester including, but not limited to, an alkyl ester such as isobutyl ester.

In some embodiments, the nucleoside compound is CEM-042 (compound 36a). In some embodiments, the nucleoside compound CEM-042 (compound 36a) has activity against viruses including, but not limited to, SARS-CoV-2, Dengue Virus, Ebola, and Chikungunya. In some embodiments, the nucleoside compound is CEM-053 (compound 36b). In some embodiments, the nucleoside compound CEM-053 (compound 36b) has activity against viruses including, but not limited to, SARS-CoV-2, Dengue Virus, and Ebola.

### Compositions Comprising the Nucleoside Compounds Described Herein

The nucleoside compounds described herein may be administered in various ways and in various forms. In some embodiments, the nucleoside compounds described herein may be administered systemically, orally (including buccal or sublingual), topically, by inhalation (or spray) or by injection (e.g., intravenously, intramuscularly, subcutaneously, intravascularly, intrathecally, intradermally, intra-arterially). For the injections, the nucleoside compounds are generally present in the form of liquid suspensions, which can be injected by means of syringes or infusions, for example. In this regard, the nucleoside compounds described herein are generally dissolved in solutions that can be buffered, isotonic, physiological, and/or saline, and which are compatible with pharmaceutical use and known to those skilled in the art.

Accordingly, in a second aspect, a composition formulated for therapeutic use is described, wherein the composition comprises, consists of, or consists essentially of at least one nucleoside compound of the first aspect, and at least one pharmaceutically acceptable carrier, excipient, or diluent. The pharmaceutical composition will typically be formulated in a format suitable for administration to the subject, for example, as a syrup, elixir, tablet, troche, lozenge, hard or soft capsule, pill, suppository, oily or aqueous suspension, dispersible powder or granule, emulsion, injectable, solution, sustained release formulation, or aerosol. Some examples of acceptable excipients are those that are non-toxic, will aid administration to the patient, and do not adversely affect the therapeutic benefit of the nucleoside compound. Such excipient may be a solid, liquid, semi-solid or, in the case of an aerosol composition, a gaseous excipient, that is generally understood by one of skill in the art. In some embodiments, solid pharmaceutical excipients include, but are not limited to, starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients include, but are not limited to, glycerol, propylene glycol, water, ethanol, and various oils, including those of petroleum, animal, vegetable, or synthetic origin, e.g., peanut oil, soybean oil, mineral oil, sesame oil, etc. Preferred liquid carriers, particularly for injectable solutions, include, but are not limited to, water, saline, aqueous dextrose, and glycols. Liquid compositions may contain one or more agents or carriers chosen from dispersants, solubilizing agents, stabilizers, preservatives, and any combination thereof. Agents or carriers which can be used in liquid and/or injectable formulations include, but are not limited to, methylcellulose, hydroxymethylcellulose, carboxymethylcellulose, polysorbate 80, mannitol, gelatin, lactose, vegetable oils, and acacia. Other suitable pharmaceutical excipients and their formulations are described in Remington's Pharmaceutical Sciences, edited by E. W. Martin (Mack Publishing Company, 18th ed., 1990).

The amount of nucleoside compound of the first aspect in the composition can vary within the full range employed by those skilled in the art. In some embodiments, a composition may contain, on a weight percent (wt %) basis, from about 0.01-99.99 wt % of a nucleoside compound, based on the total weight of the composition, with the balance being one or more suitable pharmaceutical excipients. In some embodiments, the composition comprises an amount of nucleoside compound of the first aspect of between 5 μg and 1000 mg, preferably between 1 and 500 mg, preferably between 5 and 100 mg. In some embodiments, the ratio between the amounts by weight of nucleoside compound and of pharmaceutically acceptable carrier is between 5/95 and 95/5, preferably between 20/80 and 80/20.

The nucleoside compounds may be the only active ingredients in the composition of the second aspect, or they may be combined with other active ingredients. Accordingly, in some embodiments of the second aspect, the composition may comprise, consist of, or consist essentially of at least one nucleoside compound of the first aspect, at least one other pharmaceutically active agent, e.g., at least one other medicament used for the treatment of viral infection, and at least one pharmaceutically acceptable carrier, excipient, or diluent. In some embodiments, the composition may comprise, or be combined with, one or more other antivirals or antiretrovirals (e.g., nucleoside or nucleotide and non-nucleoside inhibitors, protease inhibitors, entry inhibitors, integrase inhibitors, etc.).

### Methods of Using

In a third aspect, a method for treating and/or preventing a filoviral, flaviviral, herpesviral, alphaviral, polyomaviral, enteroviral, matonaviral, phenuiviral, Hepatitis B viral, and/or coronaviral infection is described, comprising the administration, to a patient, of an effective amount of at least one nucleoside compound of the first aspect or of a composition including same (i.e., a composition of the second aspect). In some embodiments, the nucleoside compounds, as active agents, will be administered in a therapeutically effective amount by any of the accepted modes of administration for agents that serve similar utilities. The “effective amount” will be an amount of nucleoside compound, as described herein, that would be understood by one skilled in the art to provide therapeutic benefits. The active agent can be administered once a week, two or more times per week, once a day, or more than once a day. As indicated above, all of the factors to be considered in determining the effective amount will be well within the skill of the attending clinician or other health care professional. In some embodiments, therapeutically effective amounts of nucleoside compounds, as described herein, may range from approximately 0.05 to 50 mg per kilogram body weight of the subject per day; preferably about 0.1-25 mg/kg/day, more preferably from about 0.5 to 10 mg/kg/day. Thus, for administration to a 70 kg person, the dosage range would most preferably be about 35-700 mg per day.

In some embodiments of the third aspect, a method for treating and/or preventing a viral infection in a subject is described, wherein the viral infection is caused by at least one of a coronavirus, a herpesvirus, an alphavirus, a polyomavirus, an enterovirus, a filovirus, a matonavirus, a phenuivirus, a Hepatitis B virus, and/or a flavivirus, comprising administration, to the subject, of a therapeutically effective amount of at least one nucleoside compound from the first aspect described herein, or a composition of the second aspect described herein. In some embodiments, a therapeutically effective amount of the nucleoside compounds is from 0.05 to 50 mg per kilogram body weight of the subject per day. In some embodiments, the at least one nucleoside compound is present in, and administered as, a composition, as described in the second aspect herein. In some embodiments, the viral infection is caused by a coronavirus. In some embodiments, the viral infection is caused by a herpesvirus. In some embodiments, the viral infection is caused by an alphavirus. In some embodiments, the viral infection is caused by a polyomavirus. In some embodiments, the viral infection is caused by an enterovirus. In some embodiments, the viral infection is caused by a filovirus. In some embodiments, the viral infection is caused by a matonavirus. In some embodiments, the viral infection is caused by a phenuivirus. In some embodiments, the viral infection is caused by a Hepatitis B virus. In some embodiments, the viral infection is caused by a flavivirus. In some embodiments, the method of administration is selected from systemically, orally, buccally, sublingually, topically, by inhalation, by spraying, intravenously, intramuscularly, subcutaneously, intrathecally, intradermally, intravascularly or intra-arterially.

In some embodiments, the viral infection is caused by a coronavirus selected from human coronaviruses (HCoV), Severe Acute Respiratory Syndrome Coronavirus (SARS-CoV), SARS-CoV-2, and Middle East respiratory syndrome (MERS), and mutants thereof. In some embodiments, the viral infection is caused by a herpesvirus selected from herpes simplex virus 1, herpes simplex virus 2, varicella-zoster virus, Epstein-Barr virus, cytomegalovirus, Human herpesvirus-6, Human herpesvirus-7, and Kaposi's sarcoma herpes virus. In some embodiments, the viral infection is caused by an alphavirus selected from eastern equine encephalomyelitis (EEE), Venezuelan equine encephalomyelitis (VEE), western equine encephalomyelitis (WEE), and Chikungunya. In some embodiments, the viral infection is caused by a polyomavirus. In some embodiments, the viral infection is caused by an enterovirus selected from echovirus and coxsackievirus. In some embodiments, the viral infection is caused by a filovirus selected from Zaire Ebola virus, Sudan Ebola virus, Reston Ebola virus, Cote d'Ivoire Ebola virus and Marburg virus. In some embodiments, the viral infection is caused by a matonavirus selected from Rubella, Rustrela, and Ruhugu. In some embodiments, the viral infection is caused by a phenuivirus selected from Rift Valley Fever virus. In some embodiments, the viral infection is caused by a flavivirus selected from the group consisting of yellow fever virus, Apoi virus, Aroa virus, Bagaza virus, Banzi virus, Bouboui virus, Bukalasa bat virus, Cacipacore virus, Carey Island virus, Cowbone Ridge virus, Dakar bat virus, dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Phenh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, tick-borne encephalitis virus, Tyuleniy virus, Uganda S virus, Usutu virus, Wesselsbron virus, West Nile virus, Yaounde virus, Yokose virus, Zika virus, cell fusing agent virus and Tamana bat virus.

In some embodiments of the third aspect, a method for binding to, or interacting with, natural and/or mutated polymerases of a filovirus, flavivirus, alphaviruses, polyomaviruses, enteroviruses, herpesvirus, matonavirus, phenuivirus, Hepatitis B virus, or coronavirus to induce inhibition activity of said virus is described, the method comprising administering to the subject a therapeutically effective amount of at least one nucleoside compound of the first aspect, or a composition including same (i.e., the second aspect described herein). Additional target enzymes include, but not limited to, various viral methyltransferases for all of the viruses, including the exonuclease (for CoVs), the RNA dependent RNA polymerases (RdRps) for all of the viruses, and the NiRAN (RdRp associated nucleotidyl transferase domain) for CoVs, and nucleotide-competing reverse transcriptase.

In another aspect, a use of at least one nucleoside compound of the first aspect, or a composition including same, in a medicament for medicine is described. In a more specific embodiment hereof, said use as a medicine is for the prevention or treatment of a filovirus, herpesvirus, flavivirus, alphavirus, polyomavirus, enterovirus, matonavirus, phenuivirus, Hepatitis B virus, and/or coronavirus in a subject, mammal or human. In some embodiments, a therapeutically effective amount of the at least one nucleoside compound is from 0.05 to 50 mg per kilogram body weight of the subject per day. In some embodiments, the use as a medicine is for the prevention or treatment of a coronavirus, SARS and MERS-CoV in a subject. In some embodiments, the use as a medicine is for the prevention or treatment of an infection of a COVID-19 coronavirus or a mutant thereof in a subject. In some embodiments, the use as a medicine is for the prevention or treatment of viruses including, but not limited to, filoviruses such as Ebola, Marburg and Sudan, and flaviviruses such as dengue, zika, yellow fever, and tickborne encephalitis, in a subject. In some embodiments, the use as a medicine is for the prevention or treatment of an infection of a Hepatitis B virus in a subject.

In another aspect, the manufacture of a medicament comprising at least one nucleoside compound of the first aspect, or a composition including same (i.e., the second aspect described herein), for the treatment of a coronavirus, herpesvirus, alphaviruses, enteroviruses, filovirus, matonavirus, phenuivirus, Hepatitis B virus, and/or flavivirus is described.

In another aspect, novel intermediates or prodrugs which are useful for preparing at least one nucleoside compound of the first aspect or converted to active agents in vivo is described. Prodrugs are selected and prepared in order to improve some selected property of the molecule, such as water solubility or ability to cross a membrane, temporarily. Most common (biologically labile) functional groups utilized in prodrug design include, but are not limited to, carbonates, esters, amino acyl esters, amides, carbamates, oximes, imines, ethers, or phosphates.

In a still further aspect, the present application provides for a method of treating a filovirus, flavivirus. alphaviruses, polyomaviruses, enteroviruses, herpesvirus, matonavirus, phenuivirus, Hepatitis B virus, or coronavirus in a patient, comprising administering to said patient a therapeutically effective amount of at least one nucleoside compound of the first aspect, or a composition including same (i.e., the second aspect described herein), and at least one additional therapeutic agent having antiviral properties.

In another aspect, a method for treating a viral infection comprises the administration, to a patient, of an effective amount of at least one nucleoside compound of the first aspect, or a composition including same (i.e., the second aspect described herein), is described. In some embodiments, the nucleoside compound can be a prodrug or otherwise capable of releasing the active ingredient after in vivo metabolism.

In another aspect, a method for treating a CoV viral infection is described, comprising the administration, to a patient, of an effective amount of at least one nucleoside compound of the first aspect, or a composition including same. The at least one nucleoside compound can further be a prodrug or in form of capable of releasing the active ingredient after in vivo metabolism.

In another aspect, a cell infected with a virus or to be infected with the virus is contacted with at least one nucleoside compound of the first aspect, or a composition including same (i.e., the second aspect described herein), wherein the virus is selected from a filovirus, flavivirus. alphaviruses, polyomaviruses, enteroviruses, herpesvirus, matonavirus, phenuivirus, Hepatitis B virus, or coronavirus. In some embodiments, the amount of the at least one nucleoside compound used is from about 1 μg/ml to about 40 μg/ml, and more preferably, from about 3 sg/ml to about 20 μg/ml.

In another aspect, methods for synthesis, analysis, separation, isolation, purification, characterization, and testing of the compounds of the first aspect are provided.

The features and advantages of the invention are more fully illustrated by the following non-limiting examples, wherein all parts and percentages are by weight, unless otherwise expressly stated.

### Example 1

### Synthesis of the Intermediate Bicycle Thiophene 5:

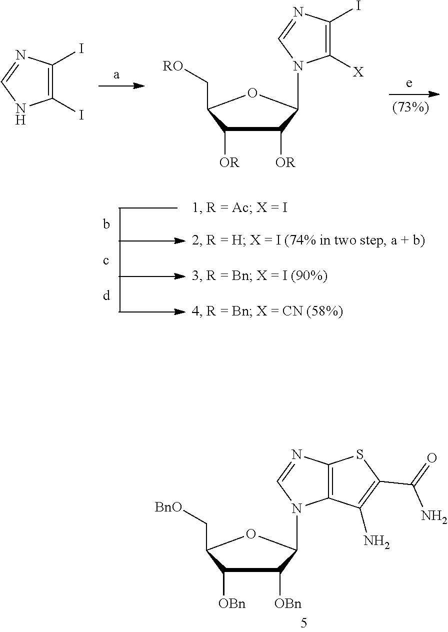

Reagents and conditions: (a) 1,2,3,5-Tetra-O-acetyl-β-D-ribofuranose, bis(trimethylsilyl)acetamide (BSA), CH₃CN, r.t. (room temperature), 4 h, and then trimethylsilyl trifluoromethanesulfonate (TMSOTf), reflux, 16 h; (b) NH₃, ethanol (EtOH), r.t., 16 h; (c) (i) NaH, tetrahydrofuran (THF), 0° C., 1 h, (ii) benzyl bromide (BnBr), Bu₄NI, r.t., 16 h; (d) (i) isopropylmagnesium chloride (IsoMgCl), THF, −78° C., 30 min; (ii) 4-methylbenzenesulfonyl cyanide, −78° C. to r.t., 16 h; (e) NH₂C(O)CH₂SH, CsCO₃, dimethylformamide (DMF), 90° C., 16 h.

### For Compounds 1-3: See Protocol: [1]-[3]

### For Compound 4: 2,3-Dibenzyloxy-5-benzyloxymethyl-1-[(5-Iodo-4-carbonitrile)imidazol-3-yl]-β-D-ribofuranose

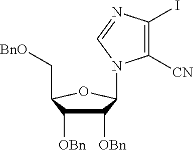

Isopropylmagnesium chloride (2.0 M in THF, 6.15 mL, 12.3 mmol, 1.15 eq.) was added dropwise over 5 min to a stirred solution of 3 (8.04 g, 11.2 mmol, 1 eq.) in anhydrous THF (75.0 mL) at −78° C. under argon. The resulting solution was then stirred for 30 min. at −78° C., and then p-toluenesulfonyl cyanide (5.05 g, 27.9 mmol, 2.5 eq.) was added. The reaction mixture was allowed to warm to room temperature and was further stirred for 16 h. After solvent removal, the crude product was purified by flash chromatography (cHex (cyclohexane)/EtOAc gradient 95:5 to 80:20, v/v) to afford compound 4 (3.39 g, 8%) as a hydroscopic white foam.

Characterization was obtained according to previously published procedure [1]:

TLC: R_{f}=0.55 (cHex/EtOAc 7:3);

¹H NMR (400 MHz, CDCl3): δ 7.82 (s, 1H), 7.40-7.19 (m, 15H), 5.86 (d, J=4.4 Hz, 1H, H), 4.68-4.62, 4.57-4.52, 4.51-4.44 (3m, 6H), 4.39-3.34 (m, 1H), 4.15-4.07 (m, 2H), 3.76 (dd, J=10.7, 3.2 Hz, 1H), 3.58 (dd, J=10.7, 2.7 Hz, 1H).

### For Compound 5: 2,3-Dibenzyloxy-5-benzyloxymethyl-1-[(5-carboxamide)-thieno[2,3-d]imidazol-3-yl]-β-D-ribofuranose

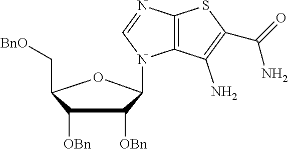

Freshly prepared thioglycolamide (1.33 g, 14.6 mmol, 2 eq.) and cesium carbonate (7.21 g, 21.9 mmol, 3 eq.) were added to a stirred solution of 4 (4.54 g, 7.31 mmol, 1 eq.) in anhydrous DMF (150 mL) under argon. The reaction mixture was heated at 90° C. for 16 h. The mixture was cooled, and the solid was filtered off. After solvent removal, the dark brown crude product was purified by flash chromatography (CH₂Cl₂/MeOH (methanol) gradient 100:0 to 96:4, v/v) to afford compound 5 (3.11 g, 73%) as a hydroscopic yellowish foam.

TLC: R_{f}=0.33 (CH₂Cl₂/MeOH 96:4);

¹H NMR (400 MHz, CDCl3): δ 7.73 (s, 1H), 7.44-7.16 (m, 13H), 7.08-7.02 (m, 2H), 6.38 (s, NH₂), 5.72 (d, J=6.5 Hz, 1H), 5.18 (s, NH₂), 4.70, 4.53, 4.48, 4.46, 4.38, 4.31 (6d, J=11.7 Hz, 6H), 4.24-4.19 (m, 1H), 4.16-4.08 (m, 2H), 3.70 (dd, J=10.7, 2.2 Hz, 1H), 3.41 (dd, J=10.7, 2.1 Hz, 1H).

¹³C NMR (101 MHz, CDCl3): δ 168.2, 146.3, 143.2, 139.6, 137.5, 136.7, 136.5, 128.9, 128.7, 128.5, 128.4, 128.4, 128.3, 128.3, 128.2, 126.1, 98.7, 88.7, 83.5, 81.2, 74.7, 73.7, 73.4, 73.1, 68.5.

### Example 2

### Synthesis of the Nucleoside Prodrug Compound 9 (CEM-015):

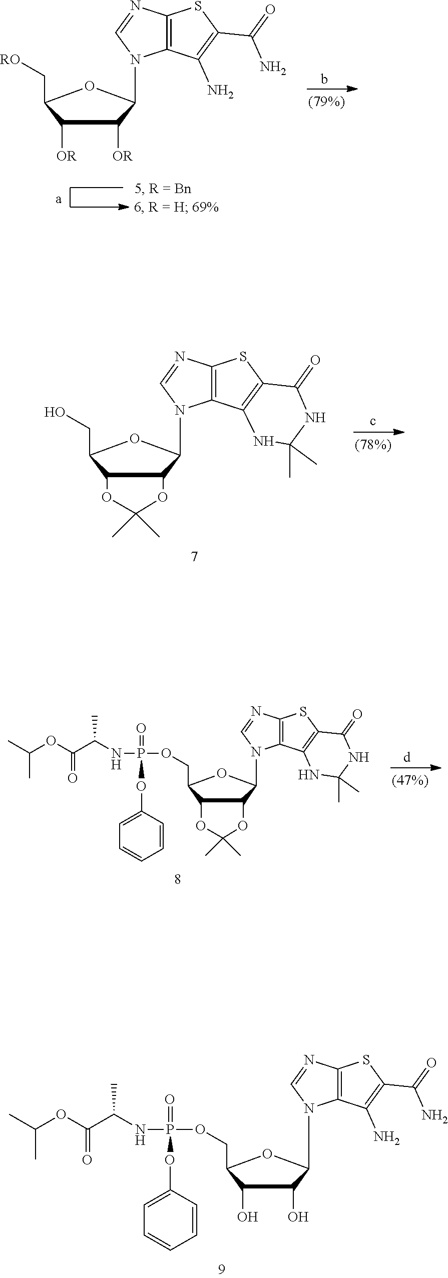

Reagents and conditions: (a) BF₃OEt₂, ethanethiol (EtSH), dichloromethane (DCM), r.t., 48 h, 69%; (c) 2,2-Dimethoxypropane, p-toluenesulfonic acid (pTsOH), DMF/acetone, r.t., 16 h, 79%; (d) t-BuMgCl, DMF, −78° C., 1 h, then N—[(S)-(2,3,4,5,6-Pentafluorophenoxy)phenoxyphosphinyl]-L-alanine 1-methylethyl ester, 55° C., 16 h, 78%; (e) trifluoroacetic acid (TFA), MeOH, 50° C., 16 h, 47%.

### For Compound 6: 1-[(5-carboxamide)-thieno[2,3-d]imidazol-3-yl]-β-D-ribofuranose 2,3,5-Triol

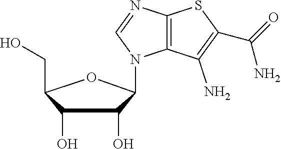

BF₃·OEt₂ (3.26 mL, 26.4 mmol, 25 eq.), and EtSH (8.90 mL, 106 mmol, 100 eq.) were added to a stirred solution of 5 (618 mg, 1.06 mmol, 1 eq.) in anhydrous DCM (25 mL) under argon. The reaction mixture was stirring for 48 h at room temperature. After solvent and excess reagents removal, the crude product was purified by flash chromatography on reversed phase (H₂O/MeCN gradient 100:0 to 80:20, v/v) and freeze-drying to afford compound (6) (223 mg, 69%) as a white solid.

¹H NMR (400 MHz, CD₃OD): δ 8.19 (s, 1H, 5.76 (d, J=7.0 Hz, 1H), 4.30 (dd, J=6.3, 3.4 Hz, 1H), 4.26-4.20 (m, 1H), 4.12-4.07 (m, 2H), 3.89 (dd, J=12.1, 2.5 Hz, 1H), 3.82 (dd, J=12.1, 2.7 Hz, 1H).

¹³C NMR (101 MHz, CD₃OD): δ 169.6, 145.5, 143.5, 139.6, 126.1, 98.6, 90.1, 86.6, 75.4, 69.1, 60.5.

Anal. Calcd for C₁₁H₁₄N₄O₅S: C, 42.03; H, 4.49; N, 17.83. Found: C, 41.59; H, 4.92; N, 16.87.

### For Compound 7: 2,3-O-isopropylidene-1-[(5-carboxamide)-thieno[2,3-d]imidazol-3-yl]-β-D-ribofuranose

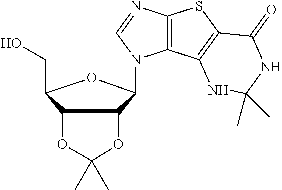

p-Toluenesulfonic acid (1.04 g, 5.48 mmol, 8 eq.) was added to a stirred solution of 6 (250 mg, 0.685 mmol, 1 eq.) in anhydrous DMF (2 mL) and 2,2-dimethoxypropane (2 ml) under argon. The reaction mixture was stirring for 16 h at room temperature. The reaction mixture was then poured into saturated NaHCO₃ solution (25 mL) and extracted with dichloromethane (3×25 mL). The combined organic layers were washed with brine, dried over Na₂SO₄ and concentrated under reduced pressure. The crude residue was purified by flash chromatography (CH₂Cl₂/MeOH gradient 100:0 to 95:5, v/v) to afford compound (7) (256 mg, 79%) as a white solid.

TLC: R_{f}=0.39 (CH₂Cl₂/MeOH 95:5);

¹H NMR (400 MHz, CD₃OD): δ 8.19 (s, 1H), 5.90 (d, J=4.8 Hz, 1H), 5.01 (dd, J=6.9, 3.4 Hz, 1H), 4.78 (dd, J=6.9, 4.8 Hz, 1H), 4.28-4.24 (m, 1H), 3.85-3.95 (m, 2H), 1.59 (s, 3H), 1.55 (s, 6H), 1.35 (s, 3H).

¹³C NMR (101 MHz, CD₃OD): δ 165.3, 151.3, 144.9, 137.8, 125.2, 116.9, 105.1, 93.0, 86.2, 85.9, 80.9, 70.5, 61.4, 28.6, 27.8, 27.4, 25.5.

### For Compound 8: Synthesis of the Protected Nucleoside Prodrug

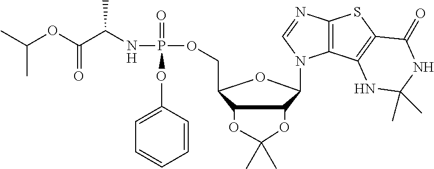

tert-Butyl magnesium chloride (315 μL, 0.315 mmol, 2.0 eq., 1 M solution in THF) was added slowly to a stirred solution of 7 (62.0 mg, 0.157 mmol, 1 eq.) in anhydrous THF/DMF (5 mL, 4;1, v/v) at −78° C. After completion of the addition, the mixture was stirred at −78° C. for 20 min. To the above mixture was added freshly prepared phosphorous reagent N—[(S)-(2,3,4,5,6-pentafluorophenoxy)phenoxyphosphinyl]-L-alanine 1-methylethyl ester (450 μl, 0.315 mmol, 2 eq., 0.7 M solution in THF) dropwise, and the resulting mixture was stirred 16 h at 55° C. After solvent removal, the crude residue was purified by flash chromatography (CH₂Cl₂/MeOH gradient 100:0 to 96:4, v/v) to afford compound 8 (81.5 mg, 78%) as a white solid.

TLC: R_{f}=0.27 (CH₂Cl₂/MeOH 97:3);

¹H NMR (400 MHz, CDCl3): δ 7.95 (s, 1H), 7.28-7.04 (m, 5H), 6.40 (s, NH), 5.88 (d, J=3.3 Hz, 1H), 5.38 (s, NH), 4.95-4.76 (m, 3H), 4.62-6.52 (m, 1H), 4.32-4.18 (m, 2H), 3.96 (t, J=10.5 Hz, NH), 3.82-3.69 (m, 1H), 1.61 (s, 3H), 1.57 (s, 3H), 1.54 (s, 3H), 1.38 (s, 3H), 1.23 (d, J=6.9 Hz, 3H), 1.14 (d, J=6.3 Hz, 2H), 1.14 (d, J=6.6 Hz, 2H).

¹³C NMR (101 MHz, CDCl₃): δ 172.9, 172.9, 163.0, 150.5, 150.4, 150.3, 135.1, 129.8, 125.2, 120.2, 120.1, 115.5, 106.9, 93.5, 84.9, 84.4, 84.3, 81.5, 69.4, 69.0, 66.1, 66.0, 50.4, 28.9, 28.6, 27.1, 25.2, 21.7, 21.6, 20.9, 20.8.

³¹P NMR (162 MHz, CDCl₃): δ 3.35.

### For Compound 9: Synthesis of the Nucleoside Prodrug

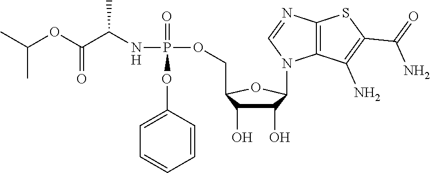

Compound 8 (108 mg, 0.163 mmol, 1 eq.) was suspended in MeOH (2 mL) and 500 μl of TFA was added. The reaction mixture was heated at 50° C. for 16 h. The solvent and the remaining TFA were then removed under reduced pressure by co-evaporating twice with toluene. Then the crude mixture was purified by flash chromatography on reversed phase (H₂O/MeCN gradient 100:0 to 70:30, v/v) and freeze-drying to afford compound 9 (44.5 mg, 47%) as a white solid.

¹H NMR (400 MHz, CD₃OD): δ 8.16 (s, 1H), 7.34-7.27 (m, 2H), 7.24-7.19 (m, 2H), 7.18-7.12 (m, 1H), 5.88-5.84 (m, 1H), 1H (signal under the CD₃OD peak), 4.43-4.30 (m, 3H), 4.30-4.26 (m, 2H), 3.89-3.80 (m, 1H), 1.29 (d, J=7.1 Hz, 3H), 1.18 (d, 6.3 Hz, 3H), 1.16 (d, 6.3 Hz, 3H).

¹³C NMR (101 MHz, CD₃OD): δ 174.3, 174.3, 170.7, 152.1, 152.0, 146.3, 143.3, 140.5, 130.8, 128.0, 126.2, 121.3, 121.3, 100.9, 91.3, 85.7, 85.6, 76.4, 71.1, 70.2, 67.2, 67.2, 21.9, 21.9, 20.5, 20.5.

³¹P NMR (162 MHz, CDCl₃): δ 4.39.

Anal. Calcd for C₂₃H₃₀N₅O₉PS: C, 47.34; H, 5.18; N, 12.00. Found: C, 46.52; H, 5.40; N, 11.62.

### Example 3

### Synthesis of the Nucleoside Prodrug Compound 14 (CEM-010):

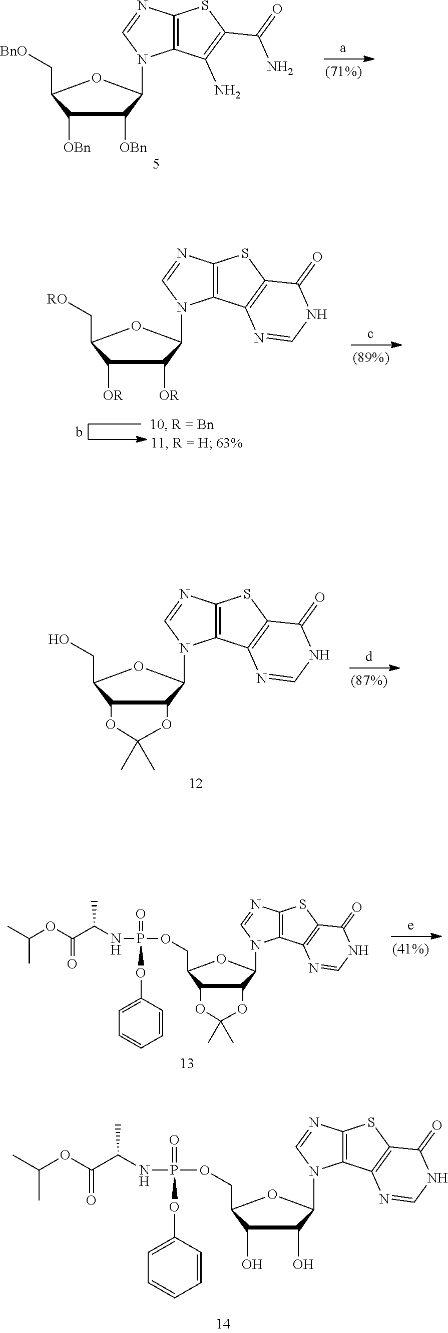

Reagents and conditions: (a) CH(OEt)₃/Ac₂O (aceticanhydride) (1:1), reflux, 16 h, 71%; (b) BF₃OEt₂, EtSH, DCM, r.t., 48 h, 63%; (c) 2,2-Dimethoxypropane, pTsOH, DMF/acetone, r.t., 16 h, 89%; (d) t-BuMgCl, DMF, −78° C., 1 h, then N-r(S)-(2,3,4,5,6-Pentafluorophenoxy)phenoxyphosphinyl-L-alanine 1-methylethyl ester, 55° C., 16 h, 87%; (e) TFA, DCM, r.t., 16 h, 41%.

### For Compound 10: 2,3-Dibenzyloxy-5-benzyloxymethyl-1-(imidazo[4′,5′:4,5]thieno[3,2-d]pyrimidin-3-yl-7-one)-β-D-ribofuranose

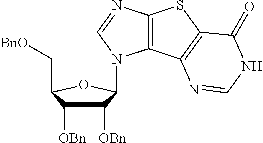

A mixture of 5 (1.00 g, 1.71 mmol), triethylorthoformate (10 mL) and acetic anhydride (10 mL) was refluxed for 6 h. After the excess solvent was evaporated, the crude residue was purified by flash chromatography (CH₂Cl₂/MeOH gradient 100:0 to 97:3, v/v) to afford compound 10 (721 mg, 71%) as a hydroscopic white foam.

TLC: R_{f}=0.16 (CH₂Cl₂/MeOH 97:3);

Characterization was obtained according to previously published procedure [1]:

¹H NMR (400 MHz, CDCl₃): δ 8.17 (s, 1H), 7.87 (s, 1H), 7.40-7.22 (in, 10H), 7.18-7.00 (m, 5H), 6.29 (d, J=5.2 Hz, 1H), 4.78-4.51 (m, 7H), 4.50-5.36 (m, 1H), 4.24 (t, J=4.7 Hz, 1H), 3.85 (dd, J=10.4, 4.8 Hz, 1H), 3.65 (dd, J=10.4, 4.2 Hz, 1H).

¹³C NMR (101 MHz, CDCl₃): δ 160.7, 151.9, 145.1, 144.6, 143.6, 137.8, 137.6, 137.3, 128.7, 128.2, 128.1, 128.0, 128.0, 127.9, 121.7, 89.0, 82.3, 79.4, 75.4, 73.6, 72.4, 72.3, 68.9.

### For Compound 11: 1-[5-(Pyrimidin-6-yl-4-one)imidazol-1-yl]-1-β-D-ribofuranose 2,3,5-Triol

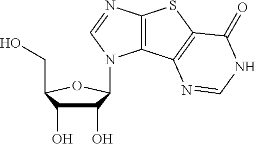

BF₃·OEt₂ (356 μL, 2.88 mmol, 8.5 eq.), and EtSH (1.47 mL, 19.9 mmol, 59 eq.) were added to a stirred solution of 10 (200 mg, 0.337 mmol, 1 eq.) in anhydrous DCM (7 mL) under argon. The reaction mixture was stirring for 48 h at room temperature. After solvent and excess reagents removal, the crude product was purified by flash chromatography on reversed phase (H₂O/MeCN gradient 100:0 to 80:20, v/v) and freeze-drying to afford compound 11 (68.4 mg, 63%) as a white solid.

Characterization was obtained according to previously published procedure [1]:

¹H NMR (400 MHz, DMSO-D₆): δ 8.59 (s, 1H), 8.27 (s, 1H), 6.07 (d, J=6.3 Hz, 1H), 5.49 (d, J=6.3 Hz, OH), 5.25 (d, J=4.3 Hz, OH), 5.15 (t, J=5.8 Hz, OH), 4.68 (q, J=6.3 Hz, 1H), 4.18-4.11 (m, 1H), 4.02-3.93 (m, 1H), 3.76-3.66 (m, 1H), 3.63-3.53 (m, 1H).

¹³C NMR (101 MHz, DMSO-D₆): δ 157.7, 148.8, 147.2, 145.5, 142.7, 128.0, 121.0, 89.0, 86.0, 74.2, 70.2, 61.5.

Anal. Calcd for C₁₂H₁₂N₄O₅S: C, 44.44; H, 3.73; N, 17.28. Found: C, 42.06; H, 4.20; N, 16.21.

### For Compound 12: 2,3-O-isopropylidene-1-[5-(Pyrimidin-6-yl-4-one)imidazol-1-yl]-1-β-D-ribofuranose

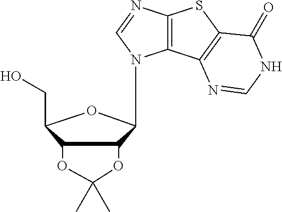

p-Toluenesulfonic acid (299 mg, 1.57 mmol, 5 eq.) and 2,2-dimethoxypropane (300 μl) were added to a stirred solution of 11 (102 mg, 0.315 mmol, 1 eq.) in anhydrous acetone (1.5 mL) and DMF (1 mL) under argon. The reaction mixture was stirring for 16 h at room temperature, then was quenched with triethylamine (300 μL). After solvent removal, the crude residue was purified by flash chromatography (CH₂Cl₂/MeOH gradient 100:0 to 95:5, v/v) to afford compound 12 (102 mg, 89%) as a white solid.

TLC: R_{f}=0.49 (CH₂Cl₂/MeOH 95:5);

¹H NMR (400 MHz, CD₃OD): δ 8.45 (s, 1H), 8.21 (s, 1H), 6.23 (d, J=4.1 Hz, 1H), 5.30 (dd, J=6.6, 4.1 Hz, 1H), 5.09 (dd, J=6.6, 3.1 Hz, 1H), 4.36-4.29 (m, 1H), 3.83 (dd, J=12.0, 4.0 Hz, 1H), 3.77 (dd, J=12.0, 4.7 Hz, 1H), 1.63 (s, 3H), 1.38 (s, 3H).

¹³C NMR (101 MHz, CD₃OD): δ 160.3, 151.5, 147.7, 146.8, 144.5, 128.9, 123.3, 116.1, 93.4, 87.3, 85.9, 82.3, 63.2, 27.5, 25.6.

### For Compound 13: Synthesis of the Protected Nucleoside Prodrugs

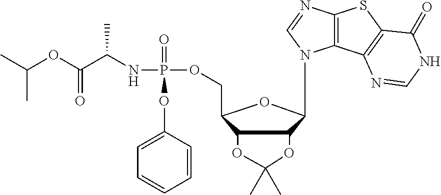

tert-Butyl magnesium chloride (390 μL, 0.390 mmol, 1.5 eq., 1 M solution in THF) was added slowly to a stirred solution of 12 (94.5 mg, 0.260 mmol, 1 eq.) in anhydrous DMF (3.7 mL) at −78° C. After completion of the addition, the mixture was stirred at −78° C. for 20 min. To the above mixture was added freshly prepared phosphorous reagent N—[(S)-(2,3,4,5,6-pentafluorophenoxy)phenoxyphosphinyl]-L-alanine 1-methylethyl ester (743 μl, 0.520 mmol, 2 eq., 0.7 M solution in DMF) dropwise, and the resulting mixture was stirred 16 h at 55° C. A saturated aqueous solution of NH₄Cl was added and the aqueous phase was extracted with DCM (20 mL×3). The combined organic layers were washed with brine, dried over Na₂SO₄ and concentrated under reduced pressure. The crude residue was purified by flash chromatography (CH₂Cl₂/MeOH gradient 100:0 to 96:4, v/v) to afford compound 13 (143 mg, 87%) as a white solid.

TLC: R_{f}=0.31 (CH₂Cl₂/MeOH 96:4);

¹H NMR (400 MHz, CDCl₃): δ 8.12 (s, 1H), 7.97 (s, 1H), 7.28-6.97 (m, 5H), 5.98 (d, J=3.7 Hz, 1H), 5.29 (dd, J=6.8, 3.7 Hz, 1H), 5.03 (dd, J=6.8, 3.4 Hz, 1H), 4.85 (p, J=6.3 Hz, 1H), 4.67-4.51 (m, 1H and NH), 4.41-4.31 (m, 1H), 4.24-4.16 (m, 1H), 3.88-3.77 (m, 1H), 1.56 (s, 3H), 1.28 (s, 3H), 1.24 (d, J=7.0 Hz, 3H), 1.10 (d, J=6.3 Hz, 3H).

¹³C NMR (101 MHz, CDCl₃): δ 173.0, 172.9, 159.7, 151.7, 150.8, 150.7, 145.7, 144.7, 143.2, 129.8, 127.4, 125.1, 122.5, 120.3, 120.3, 115.4, 92.6, 84.8, 84.0, 83.9, 81.5, 69.3, 65.7, 65.7, 50.3, 27.3, 25.4, 21.7, 21.7, 20.7, 20.6.

³¹P NMR (162 MHz, CDCl₃): δ 3.7.

### For Compound 14: Synthesis of the Nucleoside Prodrug

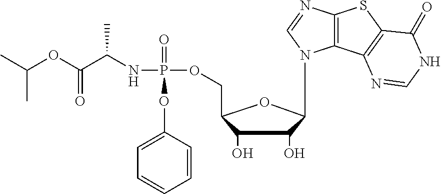

Compound 13 (143 mg, 0.234 mmol, 1 eq.) was suspended in water (3 mL) and 3 ml of TFA was added. The reaction mixture was stirred at room temperature for 6 h. The solvent and the remaining TFA were then removed under reduced pressure by co-evaporating twice with toluene. Then the crude mixture was purified by flash chromatography on reversed phase (H₂O/MeCN gradient 100:0 to 75:35, v/v) and freeze-drying to afford compound 14 (56.5 mg, 41%) as a white solid.

TLC: R_{f}=0.20 (CH₂Cl₂/MeOH 93:7);

¹H NMR (400 MHz, CD₃OD): δ 8.38 (s, 1H), 8.21 (s, 1H), 7.33-7.26 (m, 2H), 7.22-7.17 (m, 2H), 7.17-7.13 (m, 1H), 6.08 (d, J=6.2 Hz, 1H), 5.01 (t, J=5.8 Hz, 1H), 1H (signal under the CD₃OD peak), 4.55-4.50 (m, 1H), 4.43-4.38 (m, 1H), 4.38-4.34 (m, 1H), 4.29-4.24 (m, 1H), 3.81 (p, J=7.2 Hz, 1H), 1.24 (d, J=7.2 Hz, 3H), 1.15 (d, 6.3 Hz, 3H), 1.13 (d, 6.3 Hz, 3H).

¹³C NMR (101 MHz, CD₃OD): δ 174.4, 174.3, 160.4, 152.2, 152.1, 151.5, 147.6, 147.0, 144.9, 130.7, 129.2, 126.1, 123.1, 121.4, 121.3, 92.1, 85.0, 85.0, 75.3, 71.7, 70.1, 67.6, 51.5, 21.9, 21.8, 20.4, 20.4.

³¹P NMR (162 MHz, CD₃OD): δ 4.4.

Anal. Calcd for C₂₄H₂₈N₅O₉PS: C, 48.57; H, 4.76; N, 11.80. Found: C, 47.88; H, 4.86; N, 11.61.

### Example 5

### Synthesis of the Nucleoside Prodrug Compound 19 (CEM-009):

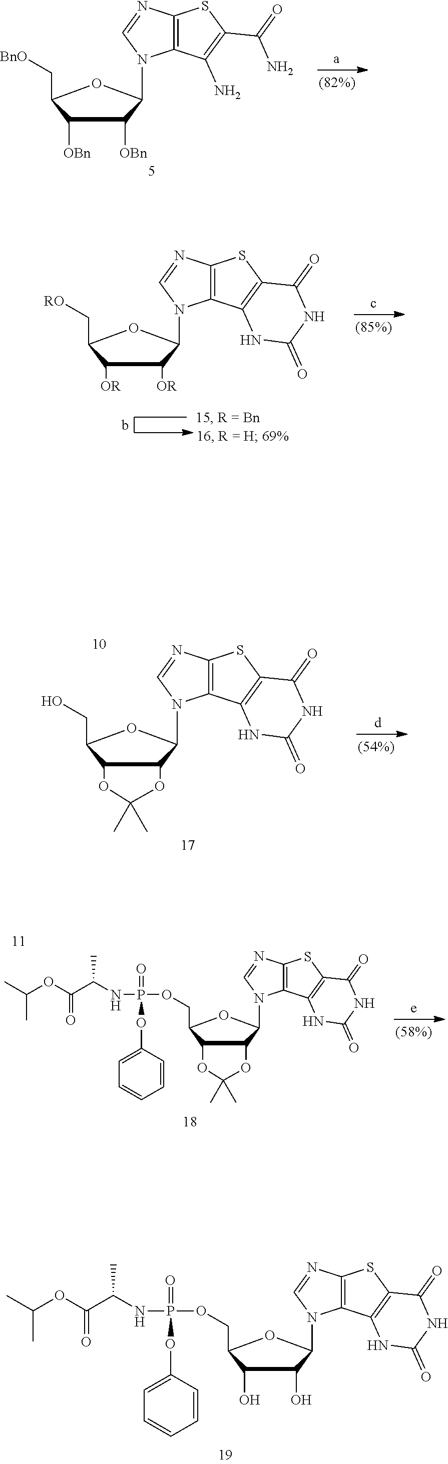

Reagents and conditions: (a) (EtO)₂CO, sodium ethoxide (NaOEt), EtOH, 90° C., 16 h, 82%; (b) BF₃OEt₂, EtSH, DCM, r.t., 48 h, 69%; (c) 2,2-Dimethoxypropane, pTsOH, DMF/acetone, r.t., 16 h, 85%; (d) t-BuMgCl, DMF, −78° C., 1 h, then N—[(S)-(2,3,4,5,6-Pentafluorophenoxy)phenoxyphosphinyl]-L-alanine 1-methylethyl ester, 55° C., 16 h, 54%; (e) TFA, DCM, r.t., 16 h, 58%.

### For Compound 15: 2,3-dibenzyloxy-5-benzyloxymethyl-1-[(5-hydroxyimidazo-[4′,5′:4,5]-thieno-[3,2-d]-pyrimidin-3-yl-7-one)]-β-D-ribofuranose

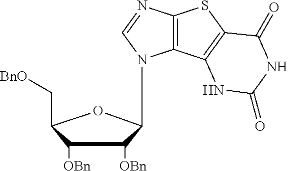

Sodium ethoxide (21 wt % in ethanol, 437 mg, 6.42 mmol, 3 eq.) was added slowly to a stirred solution of 5 (1.25 g, 2.14 mmol, 1 eq.) in diethyl carbonate (10 mL) at room temperature. The reaction mixture was stirred at 90° C. for 16 h. After the excess solvent was evaporated and the crude residue was purified by flash chromatography (CH₂Cl₂/MeOH gradient 100:0 to 97:3, v/v) to afford compound 15 (1.08 g, 82%) as a hydroscopic white foam.

TLC: R_{f}=0.29 (CH₂Cl₂/MeOH 97:3);

¹H NMR (400 MHz, CDCl₃): δ 9.57 (s, NH), 8.75 (s, NH), 7.88 (s, 1H), 7.38-7.31 (m, 6H), 7.29-7.09 (m, 7H), 6.97-6.93 (m, 2H), 5.79 (d, J=8.1 Hz, 1H), 5.02 (d, J=12.4 Hz, 1H), 4.64 (d, J=11.9 Hz, 1H), 4.53 (dd, J=22.1, 12.2 Hz, 2H), 4.43 (d, J=11.9 Hz, 1H), 4.29-4.23 (m, 2H), 3.91 (dd, J=5.7, 1.7 Hz, 1H), 3.82 (dd, J=11.1, 1.5 Hz, 1H), 3.78 (dd, J=8.1, 5.7 Hz, 1H), 3.38 (dd, J=11.1, 1.2 Hz, 1H).

¹³C NMR (101 MHz, C DCl₃): δ 159.6, 152.5, 150.7, 144.2, 137.0, 135.8, 130.2, 128.7, 128.6, 128.6, 128.5, 128.4, 128.3, 128.2, 128.1, 121.8, 110.1, 87.9, 84.3, 80.2, 74.9, 73.5, 73.0, 72.8, 67.7.

### For Compound 16: 1-[(5-Hydroxylimidazo[4′,5′:4,5]thieno[3,2-d]pyrimidin-3-yl-7-one)]-β-D-ribofuranose 2,3,5-Triol

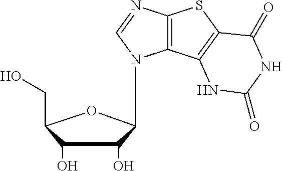

BF₃·OEt₂ (1.52 mL, 12.3 mmol, 25 eq.), and 1,2-ethanedithiol (1.24 mL, 14.7 mmol, 30 eq.) were added to a stirred solution of 15 (300 mg, 0.491 mmol, 1 eq.) in anhydrous DCM (10 mL) under argon. The reaction mixture was stirring for 48 h at room temperature. After solvent and excess reagents removal, the crude product was purified by flash chromatography on reversed phase (H₂O/MeCN gradient 100:0 to 80:20, v/v) and freeze-drying to afford compound 16 (106 mg, 69%) as a white solid.

¹H NMR (400 MHz, DMSO-D₆): δ 11.39 (s, NH), 11.30 (s, NH), 8.48 (s, 1H), 5.96-5.91 (m, 1H and OH), 5.59 (d, J=6.7 Hz, OH), 5.34 (d, J=4.2 Hz, OH), 4.13-4.00 (m, 2H), 4.02-3.95 (m, 1H), 3.73-3.63 (m, 2H).

¹³C NMR (101 MHz, DMSO-D₆): δ 159.9, 151.2, 149.9, 145.7, 131.1, 122.4, 108.3, 89.0, 86.2, 75.6, 69.7, 60.7.

Anal. Calcd for C₁₂H₁₂N₄O₆S: C, 42.35; H, 3.55; N, 16.46. Found: C, 40.40; H, 3.98; N, 15.57.

### For Compound 17: 2,3-O-isopropylidene-1-[(5-Hydroxylimidazo[4′,5′:4,5]thieno[3,2-d]pyrimidin-3-yl-7-one)]-β-D-ribofuranose

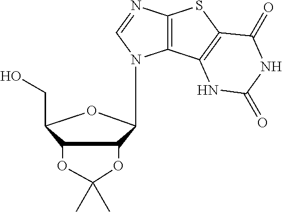

p-Toluenesulfonic acid (316 mg, 1.66 mmol, 5 eq.) and 2,2-dimethoxypropane (300 μL) were added to a stirred solution of 16 (103 mg, 0.332 mmol, 1 eq.) in anhydrous acetone (1.5 mL) and DMF (1 mL) under argon. The reaction mixture was stirring for 16 h at room temperature, then was quenched with triethylamine (300 μL). After solvent removal, the crude residue was purified by flash chromatography (CH₂Cl₂/MeOH gradient 100:0 to 95:5, v/v) to afford compound 17 (107 mg, 85%) as a white solid.

TLC: R_{f}=0.40 (CH₂Cl₂/MeOH 96:4);

¹H NMR (400 MHz, CD₃OD): δ 8.36 (s, 1H), 6.06 (d, J=4.7 Hz, 1H), 5.06 (dd, J=6.6, 2.5 Hz, 1H), 1H (signal under the CD₃OD peak), 4.49-4.44 (m, 1H), 3.84 (dd, J=11.7, 2.8 Hz, 1H), 3.79 (dd, J=11.7, 2.6 Hz, 1H), 1.64 (s, 3H), 1.39 (s, 3H).

¹³C NMR (101 MHz, CD₃OD): δ 162.2, 153.2, 152.4, 145.8, 132.7, 123.8, 116.6, 110.8, 93.8, 86.7, 85.5, 81.8, 61.8, 27.3, 25.4.

### For Compound 18: Synthesis of the Protected Nucleoside Prodrug

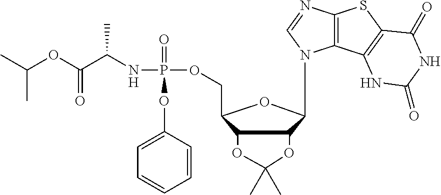

tert-Butyl magnesium chloride (242 μL, 0.242 mmol, 2 eq., 1 M solution in THF) was added slowly to a stirred solution of 17 (46.0 mg, 0.121 mmol, 1 eq.) in anhydrous DMF (2 mL) at −78° C. After completion of the addition, the mixture was stirred at −78° C. for 20 min. To the above mixture was added freshly prepared phosphorous reagent N—[(S)-(2,3,4,5,6-pentafluorophenoxy)phenoxyphosphinyl]-L-alanine 1-methylethyl ester (346 μl, 0.242 mmol, 2 eq., 0.7 M solution in DMF) dropwise, and the resulting mixture was stirred 16 h at 55° C. A saturated aqueous solution of NH₄Cl was added and the aqueous phase was extracted with DCM (20 mL×3). The combined organic layers were washed with brine, dried over Na₂SO₄ and concentrated under reduced pressure. The crude residue was purified by flash chromatography (CH₂Cl₂/MeOH gradient 100:0 to 96:4, v/v) to afford compound 18 (39.4 mg, 54%) as a white solid.

TLC: R_{f}=0.31 (CH₂Cl₂/MeOH 96:4);

¹H NMR (400 MHz, CDCl₃): δ 9.39 (s, NH), 9.22 (s, NH), 8.01 (s, 1H), 7.26-6.97 (m, 5H), 5.85 (br s, 1H), 5.00-4.79 (m, 3H), 4.68 (br s, 1H), 4.38-4.25 (m, 2H), 4.20 (t, J=10.1 Hz, NH), 3.79-3.63 (m, 1H), 1.60 (s, 3H), 1.37 (s, 3H), 1.21 (d, J=7.0 Hz, 3H), 1.12 (d, J=6.2 Hz, 6H).

¹³C NMR (101 MHz, CDCl₃): δ 172.9, 172.8, 164.8, 159.7, 152.0, 151.0, 150.4, 150.4, 142.4, 130.2, 129.8, 125.2, 120.3, 120.2, 116.2, 110.5, 93.3, 84.6, 84.6, 84.3, 81.2, 69.4, 65.9, 65.9, 50.5, 27.1, 25.3, 21.8, 21.7, 21.0, 20.9.

³¹P NMR (162 MHz, CDCl₃): δ 4.0.

### For Compound 19: Synthesis of the Nucleoside Prodrug

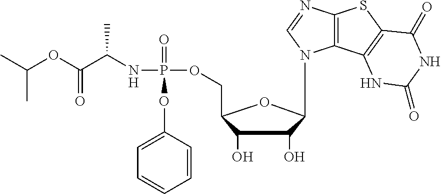

Compound 18 (72.6 mg, 0.112 mmol, 1 eq.) was suspended in water (1.5 mL) and 1.5 mL of TFA was added. The reaction mixture was stirred at room temperature for 16 h. The solvent and the remaining TFA were then removed under reduced pressure by co-evaporating twice with toluene. Then the crude mixture was purified by flash chromatography on reversed phase (H₂O/MeCN gradient 100:0 to 60:40, v/v) and freeze-drying to afford compound 19 (39.4 mg, 58%) as a white solid.

TLC: R_{f}=0.31 (CH₂Cl₂/MeOH 93:7);

¹H NMR (400 MHz, CD₃OD): δ 8.30 (s, 1H), 7.25-7.19 (m, 2H), 7.12-7.05 (m, 3H), 5.99 (d, J=5.5 Hz, 1H), 1H (signal under the CD₃OD peak), 4.50-4.33 (m, 3H), 4.32-4.20 (m, 2H), 3.75-3.64 (m, 1H), 1.22 (d, J=7.1 Hz, 3H), 1.16 (d, J=6.3 Hz, 6H).

¹³C NMR (101 MHz, CD₃OD): δ 174.2, 174.2, 162.2, 153.1, 151.9, 151.8, 151.6, 144.2, 132.7, 130.7, 126.2, 121.3, 121.3, 110.6, 92.1, 86.6, 86.5, 76.9, 72.1, 70.2, 67.3, 51.6, 21.9, 21.9, 20.5, 20.4.

³¹P NMR (162 MHz, CD₃OD): δ 4.4.

Anal. Calcd for C₂₄H₂₈N₅O₁₀PS: C, 47.29; H, 4.63; N, 11.49. Found: C, 46.58; H, 4.8; N, 11.22.

### Example 6

### Synthesis of the Nucleoside Prodrug Compound 24 (CEM-011):

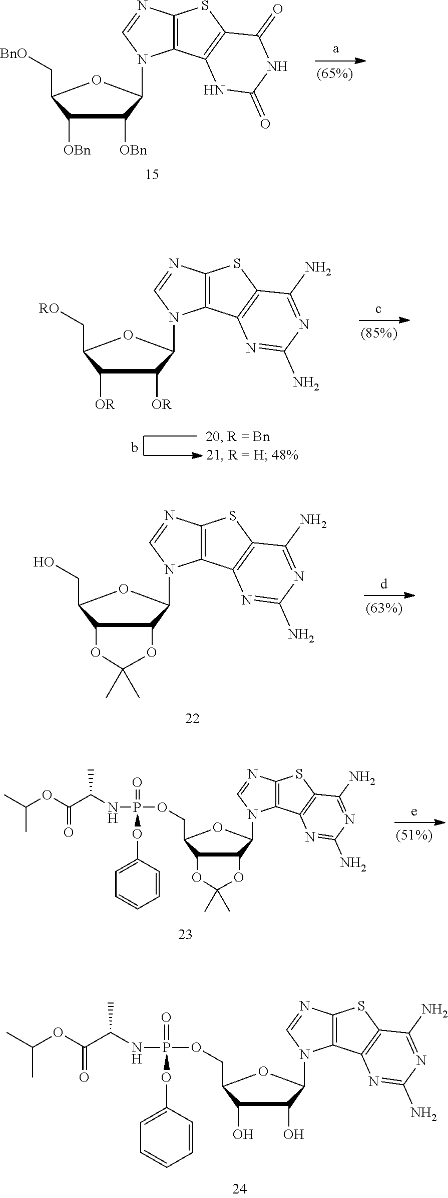

Reagents and conditions: (a) i) 2,4,6-triisopropylbenzenesulfonyl chloride (TIPBSCl), 4-dimethylaminopyridine (DMAP), triethanolamine (TEA), DCM, r.t., 16 h; ii) NH₃(g), THF, 130° C., 48 h, 65%; (b) BF₃OEt₂, EtSH, DCM, r.t., 48 h, 48%; (c) 2,2-Dimethoxypropane, pTsOH, DMF/acetone, r.t., 16 h, 85%; (d) t-BuMgCl, DMF, −78° C., 1 h, then N—[(S)-(2,3,4,5,6-Pentafluorophenoxy)phenoxyphosphinyl]-L-alanine 1-methylethyl ester, 55° C., 16 h, 63%; (e) TFA, DCM, r.t., 16 h, 51%.

### For Compound 20: 2,3-Dibenzyloxy-5-benzyloxymethyl-1-[(5,7-diaminoimidazo-[4′,5′:4,5]thieno[3,2-d]pyrimidin-3-yl)]-β-D-ribofuranose

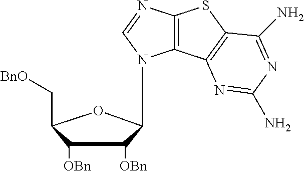

To a mixture of compound 15 (400 mg, 0.650 mmol, 1 eq.), DMAP (323 mg, 2.62 mmol, 4 eq.) and triethylamine (15 ml) in anhydrous DCM was added TIPBSCl (1.60 g, 5.24 mmol, 8 eq.) portion wise at 0° C. The reaction mixture was allowed to warm up to room temperature and was stirring for 16 h. The solvent was removed under reduced pressure and anhydrous THF (25 mL) added to the residue. The mixture was then transferred to a steel bomb and ammonia bubbled through for 20 min. The bomb was sealed and heated in an oil bath at 130° C. for 48 h. After solvent removal, the crude product was purified by flash chromatography (CH₂Cl₂/MeOH gradient 100:0 to 97:3, v/v) to afford compound 20 (258 mg, 65%) as a white solid.

TLC: R_{f}=0.24 (CH₂Cl₂/MeOH 97:3);

¹H NMR (400 MHz, CDCl₃): δ 8.05 (s, 1H), 7.32-7.16 (m, 10H), 7.11 (br s, 5H), 6.29 (d, J=4.0 Hz, 1H), 4.88 (br s, NH₂), 4.77-4.62 (m, 3H), 4.53-4.34 (m, 6H), 4.19-4.14 (m, 1H), 3.83 (dd, J=10.5, 4.3 Hz, 1H), 3.59 (dd, J=10.5, 3.7 Hz, 1H).

¹³C NMR (101 MHz, CDCl₃): δ 160.6, 158.6, 149.7, 147.9, 143.7, 137.8, 137.6, 128.7, 128.6, 128.4, 128.1, 128.0, 128.0, 128.0, 127.9, 105.6, 89.3, 81.7, 79.8, 75.4, 73.6, 72.4, 72.3, 68.7.

### For Compound 21: 1-[(5,7-diaminoimidazo-[4′,5′:4,5]thieno[3,2-d]pyrimidin-3-yl)]-β-D-ribofuranose-2,3,5-Triol

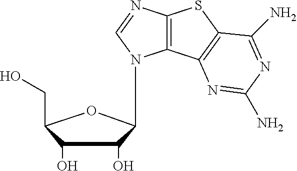

BF₃·OEt₂ (2.37 mL, 19.2 mmol, 25 eq.) and 1,2-ethanedithiol (1.94 mL, 23.0 mmol, 30 eq.) were added to a stirred solution of 20 (467 mg, 0.768 mmol, 1 eq.) in anhydrous DCM (19 mL) under argon. The reaction mixture was stirring for 48 h at room temperature. After solvent and excess reagents removal, the crude product was purified by flash chromatography on reversed phase (H₂O/MeCN gradient 100:0 to 80:20, v/v) and freeze-drying to afford compound 21 (123 mg, 48%) as a white solid.

¹H NMR (400 MHz, DMSO-D₆): δ 8.36 (s, 1H), 6.98 (s, NH₂), 6.23-6.15 (m, OH), 5.88 (d, J=7.4 Hz, 1H), 5.84 (br s, NH₂), 5.43 (d, J=6.5 Hz, OH), 5.19 (d, J=3.9 Hz, OH), 4.67-4.58 (m, 1H), 4.20-4.13 (m, 1H), 4.04-3.97 (m, 1H), 3.79-3.70 (m, 1H), 3.63-3.53 (m, 1H).

¹³C NMR (101 MHz, DMSO-D₆): δ 161.3, 159.3, 148.7, 146.2, 145.2, 126.6, 103.2, 89.0, 86.4, 74.2, 70.2, 61.7.

Anal. Calcd for C₁₂H₁₄N₆O₄S: C, 42.60; H, 4.17; N, 24.84. Found: C, 41.7; H, 4.52; N, 23.72.

### For Compound 22: 2,3-O-isopropylidene-1-[(5,7-diaminoimidazo-[4′,5′:4,5]thieno[3,2-d]pyrimidin-3-yl)]-β-D-ribofuranose

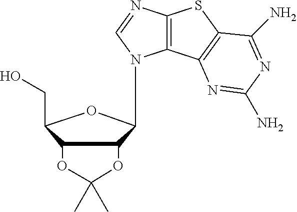

p-Toluenesulfonic acid (181 mg, 0.954 mmol, 5 eq.) and 2,2-dimethoxypropane (300 μL) were added to a stirred solution of 21 (64.5 mg, 0.191 mmol, 1 eq.) in anhydrous acetone (1.5 mL) and DMF (1 mL) under argon. The reaction mixture was stirring for 16 h at room temperature, then was quenched with triethylamine (300 μL). After solvent removal, the crude residue was purified by flash chromatography (CH₂Cl₂/MeOH gradient 100:0 to 95:5, v/v) to afford compound 22 (61.6 mg, 85%) as a white solid.

TLC: R_{f}=0.17 (CH₂Cl₂/MeOH 96:4);

¹H NMR (400 MHz, CDCl₃): δ 7.91 (s, 1H), 7.11 (s, 1H), 5.70 (d, J=6.0 Hz, 1H), 5.41-5.10 (m, 5H), 5.05-4.90 (m, 1H), 4.45 (br s, 1H), 4.08-3.97 (m, 1H), 3.90-3.77 (m, 1H), 1.62 (s, 3H), 1.35 (s, 3H).

¹³C NMR (101 MHz, CDCl₃): δ 161.3, 159.1, 151.1, 146.1, 144.2, 126.2, 115.1, 105.6, 92.7, 84.4, 83.4, 80.2, 62.2, 27.7, 25.5.

### For Compound 23: Synthesis of the Protected Nucleoside Prodrug

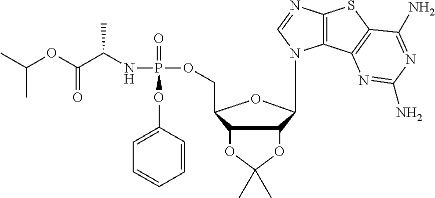

tert-Butyl magnesium chloride (288 μL, 0.288 mmol, 2 eq., 1 M solution in THF) was added slowly to a stirred solution of 22 (54.4 mg, 0.144 mmol, 1 eq.) in anhydrous DMF (2 mL) at −78° C. After completion of the addition, the mixture was stirred at −78° C. for 20 min. To the above mixture was added freshly prepared phosphorous reagent N—[(S)-(2,3,4,5,6-pentafluorophenoxy)phenoxyphosphinyl]-L-alanine 1-methylethyl ester (411 μl, 0.288 mmol, 2 eq., 0.7 M solution in DMF) dropwise, and the resulting mixture was stirred 16 h at 55° C. A saturated aqueous solution of NH₄Cl was added and the aqueous phase was extracted with DCM (20 mL×3). The combined organic layers were washed with brine, dried over Na₂SO₄ and concentrated under reduced pressure. The crude residue was purified by flash chromatography (CH₂Cl₂/MeOH gradient 100:0 to 96:4, v/v) to afford compound 23 (59.0 mg, 63%) as a white solid.

TLC: R_{f}=0.40 (CH₂Cl₂/MeOH 96:4);

¹H NMR (400 MHz, CD₃OD): δ 8.23 (s, 1H), 7.33-7.25 (m, 2H), 7.20-7.11 (m, 3H), 6.13 (d, J=3.8 Hz, 1H), 5.58 (dd, J=6.7, 3.8 Hz, 1H), 5.28 (dd, J=6.7, 2.9 Hz, 1H), 1H (signal under the CD₃OD peak), 4.63-4.54 (m, 1H), 4.44-4.39 (m, 1H), 4.26-4.17 (m, 1H), 3.93-3.79 (m, 1H), 1.60 (s, 3H), 1.36 (s, 3H), 1.25 (d, J=7.1 Hz, 3H), 1.13 (d, J=6.2 Hz, 6H).

¹³C NMR (101 MHz, CD₃OD): δ 174.4, 174.4, 163.0, 160.8, 152.1, 152.0, 150.5, 148.6, 145.8, 130.7, 128.0, 126.1, 121.4, 121.4, 116.1, 105.9, 93.8, 85.7, 85.2, 85.1, 82.9, 70.1, 67.3, 67.3, 51.6, 27.5, 25.6, 21.9, 21.8, 20.4, 20.3.

³¹P NMR (162 MHz, CD₃OD): δ 4.8.

### For Compound 24: Synthesis of the Nucleoside Prodrug

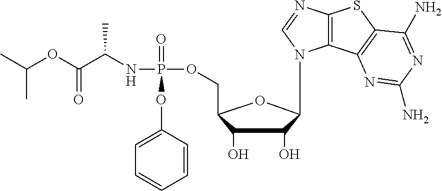

A solution of 23 (51.0 mg, 0.00788 mmol, 1 eq.) in anhydrous DCM (2 mL) was added 400 μL of TFA. The reaction mixture was stirred at room temperature for 16 h. The solvent and the remaining TFA were then removed under reduced pressure by co-evaporating twice with toluene. Then the crude mixture was purified by flash chromatography (CH₂Cl₂/MeOH[10% NH₄OH] gradient 100:0 to 90:10, v/v) to afford compound 24 (24.2 mg, 51%) as a white solid.

¹H NMR (400 MHz, CD₃OD): δ 8.22 (s, 1H), 7.34-7.08 (m, 5H), 6.00 (d, J=6.3 Hz, 1H), 5.15 (t, J=5.8 Hz, 1H), 1H (signal under the CD₃OD peak), 4.72-4.65 (m, 1H), 4.38 (dd, J=5.3, 2.6 Hz, 1H), 4.37-4.26 (m, 1H), 4.33-4.19 (m, 1H), 3.91-3.77 (m, 1H), 1.26 (d, J=7.1 Hz, 3H), 1.15 (d, J=6.3 Hz, 6H).

¹³C NMR (101 MHz, CD₃OD): δ 174.4, 174.3, 162.8, 160.9, 152.1, 152.1, 150.4, 148.3, 145.6, 130.7, 128.1, 126.1, 121.4, 121.4, 105.6, 92.5, 85.2, 85.1, 75.5, 72.4, 70.1, 67.2, 67.2, 51.6, 21.9, 21.8, 20.4, 20.4.

³¹P NMR (162 MHz, CD₃OD): δ 4.7.

Anal. Calcd for C₂₄H₃₀N₇O₈PS: C, 47.44; H, 4.98; N, 16.14. Found: C, 46.67; H, 5.22; N, 15.58.

### Example 7

### Synthesis of the Nucleoside Prodrug Compound 29 (CEM-012):

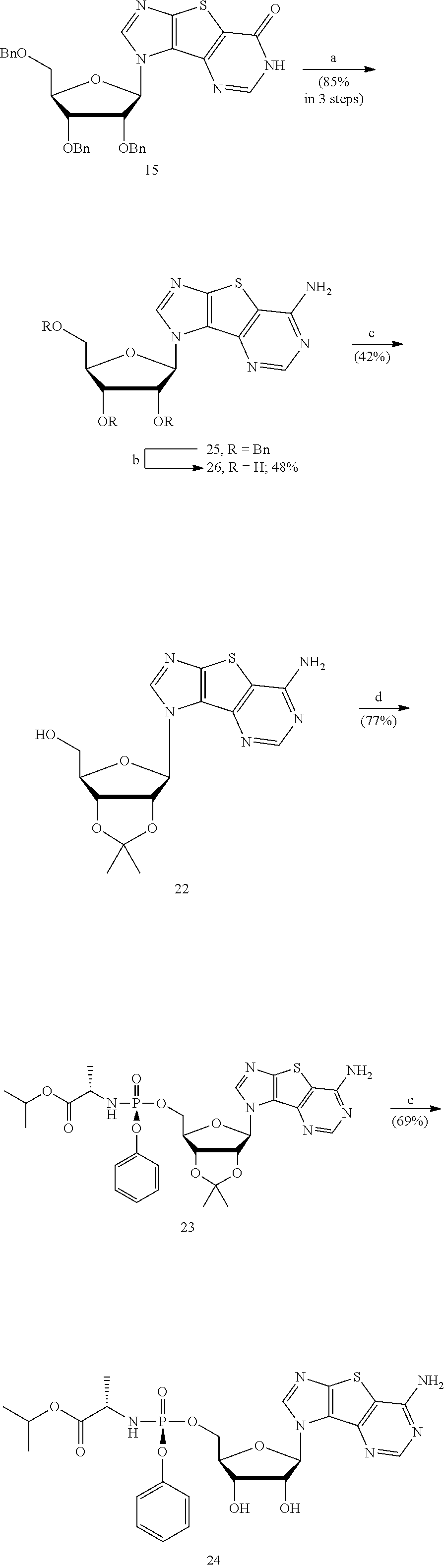

Reagents and conditions: (a) i) phosphorus pentasulfide (P₂S₅), pyridine (py.), 125° C., 16 h, 53%; ii) MeI, K₂CO₃, MeOH, r.t., 15 min., 90%; iii) NH₃(g), THF, 160° C., 60 h, 86%; (b) BF₃OEt₂, EtSH, DCM, r.t., 48 h, 48%; (c) 2,2-Dimethoxypropane, pTsOH, DMF, r.t., 16 h, 42%; (d) t-BuMgCl, DMF, −78° C., 1 h, then N—[(S)-(2,3,4,5,6-Pentafluorophenoxy)phenoxyphosphinyl]-L-alanine 1-methylethyl ester, 55° C., 16 h, 77%; (e) TFA, DCM, r.t., 16 h, 69%.

### For Compounds 10, and 25-26: See Protocol in [1]

### For Compound 27: 2,3-O-isopropylidene-1-[(7-Aminoimidazo[4′,5′:4,5]thieno[3,2-d]pyrimidin-3-yl)]-β-D-ribofuranose

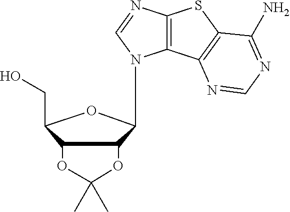

p-Toluenesulfonic acid (0.318 mg, 1.67 mmol, 5 eq.) was added to a stirred solution of 26 (108 mg, 0.334 mmol, 1 eq.) in anhydrous DMF (3 mL) and 2,2-dimethoxypropane (2 mL) under argon. The reaction mixture was stirring for 16 h at room temperature, then was quenched with triethylamine (500 μL). After solvent removal, the crude residue was purified by flash chromatography (CH₂Cl₂I/MeOH[10% NH₄OH] gradient 100:0 to 95:5, v/v) to afford compound 27 (51.0 mg, 42%) as a white solid.

TLC: R_{f}=0.30 (CH₂Cl₂/MeOH 96:4);

¹H NMR (400 MHz, CD₃OD): δ 8.36 (s, 1H), 8.35 (s, 1H), 6.09 (d, J=4.8 Hz, 1H), 5.19-5.06 (m, 2H), 4.38-4.31 (m, 1H), 3.86 (dd, J=12.3, 3.0 Hz, 1H), 3.76 (dd, J=12.3, 3.6 Hz, 1H), 1.60 (s, 3H), 1.34 (s, 3H).

¹³C NMR (101 MHz, CD₃OD): δ 160.5, 155.4, 150.7, 146.3, 145.8, 128.1, 116.1, 115.0, 93.4, 86.6, 85.3, 81.9, 63.1, 27.6, 25.6.

### For Compound 28: Synthesis of the Protected Nucleoside Prodrug

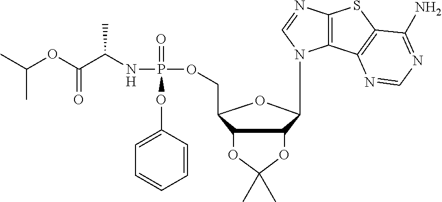

tert-Butyl magnesium chloride (338 μL, 0.338 mmol, 2 eq., 1 M solution in THF) was added slowly to a stirred solution of 27 (61.3 mg, 0.169 mmol, 1 eq.) in anhydrous DMF (2.5 mL) at −78° C. After completion of the addition, the mixture was stirred at −78° C. for 20 min. To the above mixture was added freshly prepared phosphorous reagent N—[(S)-(2,3,4,5,6-pentafluorophenoxy)phenoxyphosphinyl]-L-alanine 1-methylethyl ester (482 μl, 0.338 mmol, 2 eq., 0.7 M solution in DMF) dropwise, and the resulting mixture was stirred 16 h at 55° C. A saturated aqueous solution of NH₄Cl was added and the aqueous phase was extracted with DCM (20 mL×3). The combined organic layers were washed with brine, dried over Na₂SO₄ and concentrated under reduced pressure. The crude residue was purified by flash chromatography (CH₂Cl₂/MeOH gradient 100:0 to 95:5, v/v) to afford compound 28 (81.9 mg, 77%) as a white solid.

TLC: R_{f}=0.33 (CH₂Cl₂/MeOH 96:4);

¹H NMR (400 MHz, CDCl₃): δ 8.54 (s, 1H), 8.03 (br s, 1H), 7.34-7.06 (m, 5H), 6.10 (d, J=3.3 Hz, 1H), 5.67 (s, NH₂), 5.48-4.42 (m, 1H), 5.21-5.15 (m, 1H), 4.91 (p, J=6.2 Hz, 1H), 4.45-4.42 (m, 2H), 4.38-4.30 (m, 1H), 3.96-3.84 (m, 1H), 3.75 (t, J=10.3 Hz, NH), 1.62 (s, 3H), 1.36 (s, 3H), 1.24 (d, J=7.0 Hz, 3H), 1.16 (d, J=6.2 Hz, 3H), 1.15 (d, J=6.2 Hz, 3H).

¹³C NMR (101 MHz, CDCl₃): δ 173.0, 172.9, 157.7, 153.8, 150.7, 150.6, 150.2, 150.1, 146.0, 129.8, 129.7, 125.0, 120.3, 120.3, 115.4, 114.4, 92.5, 85.0, 84.5, 84.4, 81.3, 69.3, 66.5, 66.4, 50.3, 27.3, 25.4, 21.7, 21.7, 21.1, 21.0.

³¹P NMR (162 MHz, CDCl₃): δ 3.2.

### For Compound 29: Synthesis of the Nucleoside Prodrug

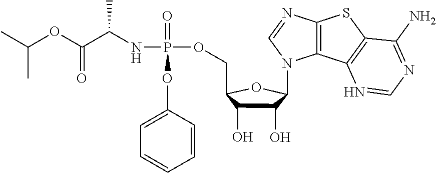

A solution of 28 (41.6 mg, 0.00658 mmol, 1 eq.) in anhydrous DCM (2 mL) was added 200 μL of TFA. The reaction mixture was stirred at room temperature for 16 h. The solvent and the remaining TFA were then removed under reduced pressure by co-evaporating twice with toluene. Then the crude mixture was purified by flash chromatography on reversed phase (H₂O/MeCN gradient 100:0 to 40:60, v/v) and freeze-drying to afford compound 29 (27.1 mg, 69%) as a white solid.

¹H NMR (400 MHz, CD₃OD): δ 8.40 (s, 1H), 8.32 (s, 1H), 7.29-7.04 (m, 5H), 6.10 (d, J=5.9 Hz, 1H), 5.01 (t, J=5.7 Hz, 1H), 4.90-4.79 (m, 1H), 4.60-4.49 (m, 1H), 4.42-4.27 (m, 3H), 3.86-3.74 (m, 1H), 1.23 (d, J=7.1 Hz, 3H), 1.13 (d, J=5.8 Hz, 3H), 1.11 (d, J=5.8 Hz, 3H).

¹³C NMR (101 MHz, CD₃OD): δ 174.4, 174.3, 160.3, 155.3, 152.1, 152.0, 150.2, 146.5, 146.0, 130.7, 128.7, 126.0, 121.3, 121.3, 114.9, 92.5, 85.3, 85.2, 75.6, 72.0, 70.1, 67.6, 67.6, 51.6, 21.9, 21.8, 20.5, 20.4.

³¹P NMR (162 MHz, CD₃OD): δ 4.3.

Anal. Calcd for C₂₄H₂₉N₆O₈PS: C, 48.65; H, 4.93; N, 14.18. Found: C, 47.82; H, 5.14; N, 13.77.

### Example 8

### Synthesis of the Nucleoside Prodrug Compounds 24a (CEM-011) and 24b (CEM-056):

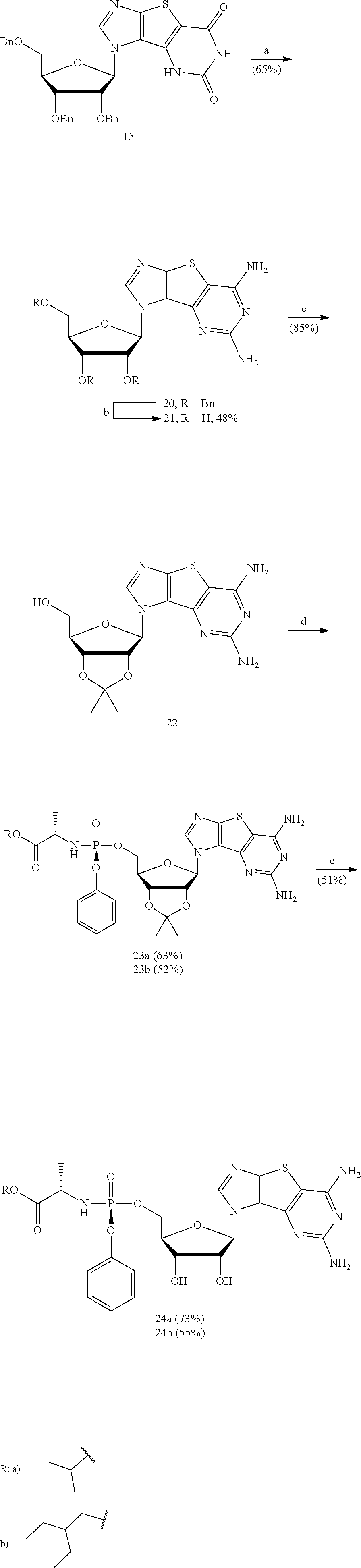

Reagents and conditions: (a) i) TIPBSCl, DMAP, TEA, DCM, r.t., 16 h; ii) NH₃(g), THF, 130° C., 48 h, 65%; (b) BF₃OEt₂, EtSH, DCM, r.t., 48 h, 48%; (c) 2,2-Dimethoxypropane, pTsOH, DMF/acetone, r.t., 16 h, 85%; (d) t-BuMgCl, DMF, −78° C., 1 h, then isopropyl ((S)-(perfluorophenoxy)(phenoxy)phosphoryl)-L-alaninate or 2-ethylbutyl ((S)-(perfluorophenoxy)(phenoxy)phosphoryl)-L-alaninate, 55° C., 16 h, 63% for 23a and 52% for 23b; (c) TFA, H₂O, DCM, rt, 8-16 h, 73% for 24a and 55% for 24b.

### For Compound 20: 2,3-Dibenzyloxy-5-benzyloxymethyl-1-[(5,7-diaminoimidazo-[4′,5′:4,5]thieno[3,2-d]pyrimidin-3-yl)]-β-D-ribofuranose

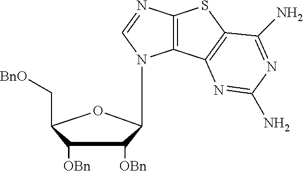

To a mixture of compound 15 (400 mg, 0.650 mmol, 1 eq.), DMAP (323 mg, 2.62 mmol, 4 eq.) and triethylamine (15 mL) in anhydrous DCM was added 2,4,6-triisopropylbenzenesulfonyl chloride (TIPBSCl, 1.60 g, 5.24 mmol, 8 eq.) portion wise at 0° C. The reaction mixture was allowed to warm up to room temperature and stirred for 16 h. The solvent was then removed under reduced pressure and anhydrous THF (25 mL) added to the resulting residue. The mixture was then transferred to a steel bomb and ammonia bubbled through for 20 min. The bomb was sealed and heated at 130° C. for 48 h. After solvent removal, the resulting crude residue was purified by flash chromatography with CH₂Cl₂/MeOH (100:0 to 97:3, v/v) to afford 20 (258 mg, 65%) as a white solid.

TLC: R_{f}=0.24 (CH₂Cl₂/MeOH 97:3);

¹H NMR (400 MHz, CDCl₃): δ=8.05 (s, 1H), 7.32-7.16 (m, 10H), 7.11 (br s, 5H), 6.29 (d, J=4.0 Hz, 1H), 4.88 (br s, NH₂), 4.77-4.62 (m, 3H), 4.53-4.34 (m, 6H), 4.19-4.14 (m, 1H), 3.83 (dd, J=10.5, 4.3 Hz, 1H), 3.59 (dd, J=10.5, 3.7 Hz, 1H);

¹³C NMR (101 MHz, CDCl₃): δ=160.6, 158.6, 149.7, 147.9, 143.7, 137.8, 137.6, 128.7, 128.6, 128.4, 128.1, 128.0, 128.0, 128.0, 127.9, 105.6, 89.3, 81.7, 79.8, 75.4, 73.6, 72.4, 72.3, 68.7;

MS (ESI): m/z 609.21 [M+H]⁺.

### For Compound 21: 1-[(5,7-diaminoimidazo-[4′,5′:4,5]thieno[3,2-d]pyrimidin-3-yl)]-β-D-ribofuranose-2,3,5-Triol

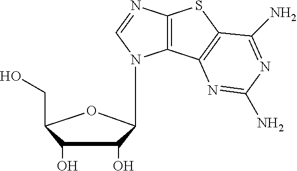

To a stirred solution of 20 (467 mg, 0.768 mmol, 1 eq.), in anhydrous DCM (19 mL), was added BF₃·Et₂O (2.37 mL, 19.2 mmol, 25 eq.) and 1,2-ethanedithiol (1.94 mL, 23.0 mmol, 30 eq.). The reaction mixture was stirred for 48 h at room temperature. After solvent and excess of reagents removal, the resulting crude residue was purified by flash chromatography on reversed phase with H₂O/MeCN (100:0 to 70:30, v/v) and freeze-dried to afford 21 (123 mg, 48%) as a white solid.

¹H NMR (400 MHz, DMSO-d₆): δ=8.36 (s, 1H), 6.98 (s, NH₂), 6.23-6.15 (m, OH), 5.88 (d, J=7.4 Hz, 1H), 5.84 (br s, NH₂), 5.43 (d, J=6.5 Hz, OH), 5.19 (d, J=3.9 Hz, OH), 4.67-4.58 (m, 1H), 4.20-4.13 (m, 1H), 4.04-3.97 (m, 1H), 3.79-3.70 (m, 1H), 3.63-3.53 (m, 1H);

¹³C NMR (101 MHz, DMSO-d₆): δ=161.3, 159.3, 148.7, 146.2, 145.2, 126.6, 103.2, 89.0, 86.4, 74.2, 70.2, 61.7;

HPLC: Method A: H₂O/CH₃CN 95:5 to 20:80 in 8 min, λ=254 nm, t_{R}=3.366 min.

### For Compound 22: 2,3-O-isopropylidene-1-[(5,7-diaminoimidazo-[4′,5′:4,5]thieno[3,2-d]pyrimidin-3-yl)]-β-D-ribofuranose

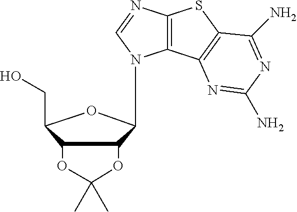

To a stirred solution of 21 (64.5 mg, 0.191 mmol, 1 eq.), in anhydrous acetone (1.5 mL) and DMF (1 mL), was added p-toluenesulfonic acid (181 mg, 0.954 mmol, 5 eq.) and 2,2-dimethoxypropane (300 μL). The reaction mixture was stirred for 16 h at room temperature and quenched with triethylamine (300 μL). After solvent removal, the resulting crude residue was purified by flash chromatography with CH₂Cl₂/MeOH (100:0 to 95:5, v/v) to afford 22 (61.6 mg, 85%) as a white solid.

TLC: R_{f}=0.17 (CH₂Cl₂/MeOH 96:4);

¹H NMR (400 MHz, CDCl₃): δ=7.91 (s, 1H), 7.11 (s, 1H), 5.70 (d, J=6.0 Hz, 1H), 5.41-5.10 (m, 5H), 5.05-4.90 (m, 1H), 4.45 (br s, 1H), 4.08-3.97 (m, 1H), 3.90-3.77 (m, 1H), 1.62 (s, 3H), 1.35 (s, 3H);

¹³C NMR (101 MHz, CDCl₃): δ=161.3, 159.1, 151.1, 146.1, 144.2, 126.2, 115.1, 105.6, 92.7, 84.4, 83.4, 80.2, 62.2, 27.7, 25.5;

MS (ESI): m/z 379.12 [M+H]⁺.

### For Compound 23a: Synthesis of the Protected Nucleoside Prodrug

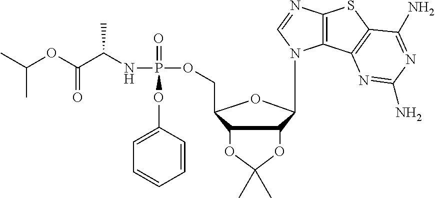

To a stirred solution of 22 (54.4 mg, 0.144 mmol, 1 eq.), in anhydrous DMF (2 mL) at −78° C., was added a solution of tert-Butyl magnesium chloride (288 μL, 0.288 mmol, 2 eq., 1 M in THF) dropwise. After 20 min., the cooling bath was removed and a freshly prepared solution of N—[(S)-(2,3,4,5,6-pentafluorophenoxy)phenoxyphosphinyl]-L-alanine 1-methylethyl ester (411 μl, 0.288 mmol, 2 eq., 0.7 M solution in DMF) was added dropwise, and the resulting mixture stirred for 16 h at 55° C. A saturated solution of aq. NH₄Cl was added and the aqueous layer extracted with DCM (20 mL×3). The combined organic layers were washed with brine, dried over Na₂SO₄ and concentrated under reduced pressure. The crude residue was then purified by flash chromatography with CH₂Cl₂/MeOH (100:0 to 96:4, v/v) to afford 23a (59.0 mg, 63%) as a white solid.

TLC: R_{f}=0.40 (CH₂Cl₂/MeOH 96:4);

¹H NMR (400 MHz, CD₃OD): δ=8.23 (s, 1H), 7.33-7.25 (m, 2H), 7.20-7.11 (m, 3H), 6.13 (d, J=3.8 Hz, 1H), 5.58 (dd, J=6.7, 3.8 Hz, 1H), 5.28 (dd, J=6.7, 2.9 Hz, 1H), 1H (signal under the CD₃OD peak), 4.63-4.54 (m, 1H), 4.44-4.39 (m, 1H), 4.26-4.17 (m, 1H), 3.93-3.79 (m, 1H), 1.60 (s, 3H), 1.36 (s, 3H), 1.25 (d, J=7.1 Hz, 3H), 1.13 (d, J=6.2 Hz, 6H);

¹³C NMR (101 MHz, CD₃OD): δ=174.4, 174.4, 163.0, 160.8, 152.1, 152.0, 150.5, 148.6, 145.8, 130.7, 128.0, 126.1, 121.4, 121.4, 116.1, 105.9, 93.8, 85.7, 85.2, 85.1, 82.9, 70.1, 67.3, 67.3, 51.6, 27.5, 25.6, 21.9, 21.8, 20.4, 20.3;

³¹P NMR (162 MHz, CD₃OD): δ=4.8;

MS (ESI): m/z 648.20 [M+H]⁺.

### For Compound 24a: Synthesis of the Nucleoside Prodrug

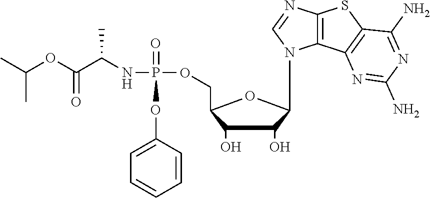

To a solution of 23a (53.0 mg, 0.0818 mmol) in DCM (2 mL) and H₂O (50 μL) was added 1.0 ml of TFA. The resulting mixture was stirred at room temperature for 4 h. The solvent and the remaining TFA were then removed under reduced pressure through multiple co-evaporation with toluene. The crude residue was then purified by flash chromatography on reversed phase with H₂O/MeCN (100:0 to 40:60, v/v) and freeze-dryed to afford 24a (36.4 mg, 73%) as a white solid.

¹H NMR (400 MHz, CD₃OD): δ=8.22 (s, 1H), 7.34-7.08 (m, 5H), 6.00 (d, J=6.3 Hz, 1H), 5.15 (t, J=5.8 Hz, 1H), 1H (signal under the CD₃OD peak), 4.72-4.65 (m, 1H), 4.38 (dd, J=5.3, 2.6 Hz, 1H), 4.37-4.26 (m, 1H), 4.33-4.19 (m, 1H), 3.91-3.77 (m, 1H), 1.26 (d, J=7.1 Hz, 3H), 1.15 (d, J=6.3 Hz, 6H). ¹³C NMR (101 MHz, CD₃OD): δ=174.4, 174.3, 162.8, 160.9, 152.1, 152.1, 150.4, 148.3, 145.6, 130.7, 128.1, 126.1, 121.4, 121.4, 105.6, 92.5, 85.2, 85.1, 75.5, 72.4, 70.1, 67.2, 67.2, 51.6, 21.9, 21.8, 20.4, 20.4. ³¹P NMR (162 MHz, CD₃OD): δ=4.7. HRMS (ESI) m/z 608.16741 (M+H)⁺ (C₂₄H₃₁N₇O₈PS⁺ requires 608.16869). HRMS-ESI (m/z): calcd for C₂₄H₃₁N₇O₈PS [M+H]⁺ 608.16869. found 608.16741. HPLC: Method A: H₂O/CH₃CN 95:5 to 20:80 in 8 min, λ=254 nm, t_{R}=5.441 min.

### For Compound 23b: Synthesis of the Protected Nucleoside Prodrug

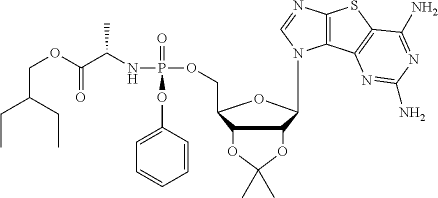

To a stirred solution of 22 (57.0 mg, 0.151 mmol, 1 eq.) in anhydrous DMF (2 mL) at −78° C. was slowly added tert-Butyl magnesium chloride (366 μL, 0.256 mmol, 1.5 eq., 1 M solution in THF). The resulting mixture was then stirred at −78° C. After 20 min., the cooling bath was removed and a freshly prepared solution of N—[(S)-(2,3,4,5,6-pentafluorophenoxy)phenoxyphosphinyl]-L-alanine 1-methylethyl ester (226 μl, 0.226 mmol, 1.7 eq., 0.7 M solution in DMF) was added dropwise, and the resulting mixture stirred for 16 h at 55° C. A saturated solution of aq. NH₄Cl was added and the aqueous layer extracted with DCM (20 mL×3). The combined organic layers were washed with brine, dried over Na₂SO₄ and concentrated under reduced pressure. The crude residue was then purified by flash chromatography with CH₂Cl₂/MeOH (100:0 to 90:10, v/v) to afford 23b (53.6 mg, 52%) as a white solid.

TLC: R_{f}=0.37 (CH₂Cl₂/MeOH 96:4);

¹H NMR (400 MHz, CDCl₃) δ=7.84 (s, 1H), 7.37-7.22 (m, 4H), 7.22-7.12 (m, 1H), 5.97 (s, 2H), 5.83 (d, J=4.4 Hz, 1H), 5.55 (dd, J=6.8, 4.4 Hz, 1H), 5.29 (s, 2H), 5.19-5.04 (m, 2H), 4.98-4.84 (m, 1H), 4.40 (ddd, J=9.8, 4.6, 2.4 Hz, 1H), 4.11-3.95 (m, 4H), 1.59 (s, 3H), 1.55-1.44 (m, 1H), 1.43-1.27 (m, 7H), 1.28 (s, 3H), 0.86 (td, J=7.5, 1.2 Hz, 6H);

¹³C NMR (101 MHz, CDCl₃) δ=174.8, 174.7, 161.9, 158.9, 150.7, 150.7, 150.2, 147.9, 144.0, 129.8, 126.6, 125.1, 120.5, 120.4, 115.0, 105.1, 93.1, 84.2, 83.0, 82.9, 82.4, 67.8, 64.9, 50.4, 40.3, 27.5, 25.4, 23.2, 20.9, 20.8, 11.0;

³¹P NMR (162 MHz, CDCl₃) δ=4.2;

MS (ESI): m/z 690.24 [M+H]⁺.

### For Compound 24b: Synthesis of the Nucleoside Prodrug

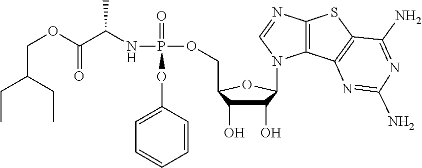

To a solution of 23b (57.6 mg, 0.0835 mmol) in DCM (2 mL) and H₂O (50 μL) was added 1.0 ml of TFA. The resulting mixture was stirred at room temperature for 4 h. The solvent and the remaining TFA were then removed under reduced pressure through multiple co-evaporation with toluene. The crude residue was then purified by flash chromatography on reversed phase with H₂O/MeCN (100:0 to 40:60, v/v) and freeze-dryed to afford 24b (30.0 mg, 55%) as a white solid.

¹H NMR (400 MHz, CD₃OD) δ=8.23 (s, 1H), 7.30 (t, J=7.8 Hz, 2H), 7.24-7.09 (m, 3H), 6.00 (d, J=6.3 Hz, 1H), 5.14 (t, J=5.8 Hz, 1H), 4.70 (dt, J=10.9, 6.3 Hz, 1H), 4.41-4.33 (m, 1H), 4.35-4.28 (m, 1H), 4.24 (dt, J=10.3, 5.0 Hz, 1H), 4.03-3.93 (m, 1H), 3.94-3.80 (m, 2H), 1.48-1.33 (m, 1H), 1.26 (dd, J=9.7, 5.0 Hz, 7H), 0.81 (t, J=7.4 Hz, 6H);

¹³C NMR (101 MHz, CD₃OD) δ=174.9, 174.9, 162.8, 160.9, 152.1, 152.1, 150.4, 148.3, 145.5, 130.7, 128.1, 126.1, 121.4, 121.4, 105.6, 92.5, 85.2, 85.1, 75.5, 72.4, 68.1, 67.2, 51.5, 41.6, 24.1, 20.5, 20.4, 11.3, 11.2;

³¹P NMR (162 MHz, CD₃OD) δ=4.6;

HRMS-ESI (m/z): calcd for C₂₇H₃₇N₇O₈PS [M+H]⁺ 650.21564. found 650.21442.

HPLC: Method A: H₂O/CH₃CN 95:5 to 20:80 in 8 min, λ=254 nm, t_{R}=5.948 min.

### Example 9

### Synthesis of the Nucleoside Compound 34 (CEM-024):

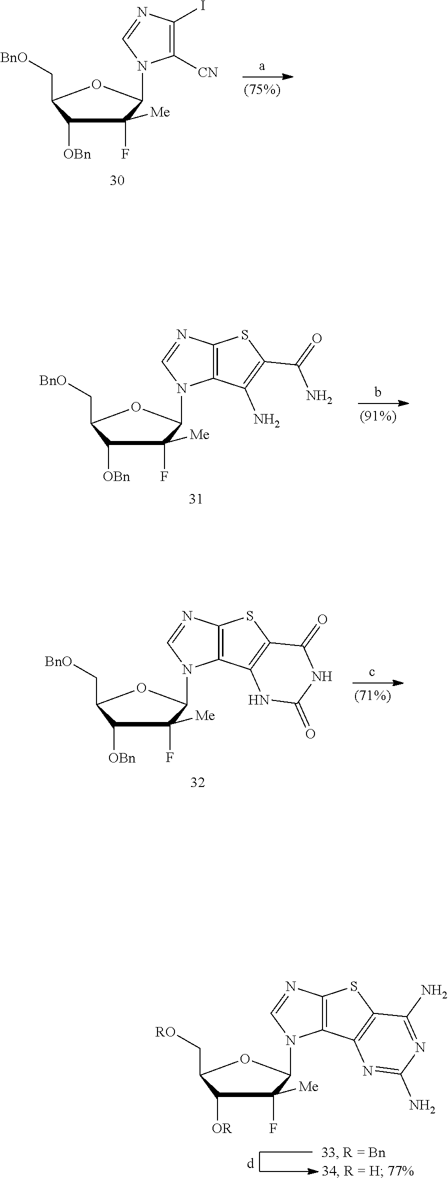

Reagents and conditions: (a) NH₂C(O)CH₂SH, Cs₂CO₃, DMF, 90° C., 16 h, 75%; (b) (EtO)₂CO, NaOEt, EtOH, 90° C., 16 h, 91%; (c) i) TIPBSCl, DMAP, TEA, DCM, rt, 16 h; ii) NH₃, THF, 130° C., 48 h, 71%; (d) BF₃·Et₂O, EtSH, DCM, rt, 48 h, 77%.

### For Compound 30: 3′,5′-Dibenzyloxy-2′-deoxy-2′-fluoro-2′-C-methyl-1′-[(5-iodo-4-carbonitrile)-imidazole-3-yl]-β-D-ribofuranose: See Protocol in [4]

### For Compound 31: 3′,5′-Dibenzyloxy-2′-deoxy-2′-fluoro-2′-C-methyl-1′-[(5-carboxamide)[2,3-d]imidazole-3-yl]-β-D-ribofuranose)

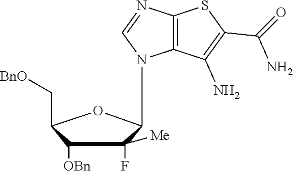

To a solution of 30 (1.13 g, 2.07 mmol, 1 eq.) in anhydrous DMF (40 mL) was added a freshly prepared thioglycolamide (565 mg, 6.21 mmol, 3 eq.) and cesium carbonate (2.04 g, 6.21 mmol, 3 eq.). The resulting mixture was heated at 90° C. for 16 h. After cooling down, the solid was filtered off and solvent removed under reduced pressure. The resulting dark brown crude residue was then purified by flash chromatography with CH₂Cl₂/MeOH (100:0 to 97:3, v/v) to afford 31 (800 mg, 75%) as a yellowish foam.

TLC: R_{f}=0.57 (CH₂Cl₂/MeOH 97:3);

¹H NMR (400 MHz, CDCl₃) δ=7.84 (s, 1H), 7.44-7.27 (m, 10H), 6.46 (s, NH₂), 5.90 (d, J=18.3 Hz, 1H), 5.21 (s, NH₂), 4.77 (d, J=11.5 Hz, 1H), 4.58 (d, J=11.6 Hz, 1H), 4.53 (d, J=11.6 Hz, 1H), 4.46 (d, J=11.7 Hz, 1H), 4.14-4.07 (m, 2H), 3.91 (d, J=11.1 Hz, 1H), 3.64 (d, J=11.0 Hz, 1H), 1.18 (d, J=23.5 Hz, 1H).

### For Compound 32: 3′,5′-Dibenzyloxy-2′-deoxy-2′-fluoro-2′-C-methyl-1′-[(5-hydroxylimidazo-[4′,5′:4,5]-thieno-[3,2-d]-pyrimidin-3-yl-7-one]-1-β-D-ribofuranose

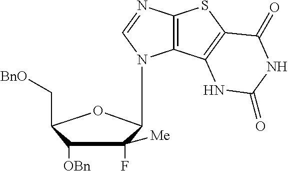

To a solution of 31 (1.47 g, 2.87 mmol, 1 eq.) in diethyl carbonate (18 mL), was added sodium ethoxide (21 wt % in ethanol, 489 mg, 7.18 mmol, 2.5 eq.) at room temperature. The resulting mixture was stirred at 90° C. for 16 h. After solvent removal the resulting crude residue was purified by flash chromatography with CH₂Cl₂/MeOH (100:0 to 95:5, v/v) to afford 32 (1.40 g, 91%) as a white solid.

TLC: R_{f}=0.5 (CH₂Cl₂/MeOH 92:8);

¹H NMR (400 MHz, CDCl₃): δ=9.81 (s, NH), 9.55 (br s, NH), 7.95 (s, 1H), 7.39-7.19 (m, 10H), 6.01 (d, J=14.2 Hz, 1H), 5.08 (d, J=12.5 Hz, 1H), 4.68 (d, J=11.3 Hz, 1H), 4.58 (d, J=12.5 Hz, 1H), 4.35 (d, J=11.4 Hz, 1H), 4.22-4.14 (m, 1H), 3.99 (dd, J=11.2, 2.0 Hz, 1H), 3.83 (t, J=5.7 Hz, 1H), 3.61-3.53 (m, 1H), 1.08 (d, J=23.2 Hz, 3H);

¹³C NMR (101 MHz, CDCl₃): δ=160.1, 151.9, 151.1, 143.2, 137.1, 136.9, 130.2, 128.9, 128.7, 128.5, 128.4, 128.2, 122.9, 110.9, 98.5, 96.5, 91.9, 91.5, 81.8, 79.0, 78.9, 73.7, 73.7, 73.6, 65.4, 19.1, 18.9;

¹⁹F NMR (376 MHz, CDCl₃) δ=−164.3, −164.4, −164.4, −164.5, −164.5.

### For Compound 33: 3′,5′-Dibenzyloxy-2′-deoxy-2′-fluoro-2′-C-methyl-1′-[(5,7-diaminoimidazo-[4′,5′:4,5]thieno[3,2-d]pyrimidin-3-yl)]-β-D-ribofuranose

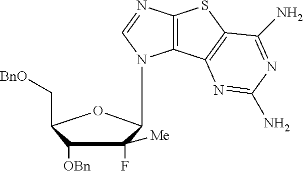

To a mixture of compound 32 (700 mg, 1.31 mmol, 1 eq.), DMAP (638 mg, 5.22 mmol, 4 eq.) and triethylamine (13 ml) in anhydrous DCM (26 ml) was added 2,4,6-triisopropylbenzenesulfonyl chloride (TIPBSCl, 2.37 g, 7.84 mmol, 6 eq.) portion wise at 0° C. The reaction mixture was allowed to warm up to room temperature and while stirring for 16 h. The solvent was then removed under reduced pressure and anhydrous THF (20 mL) added to the resulting residue. The mixture was then transferred to a steel bomb and ammonia bubbled through for 20 min. The bomb was sealed and heated at 130° C. for 48 h. After solvent removal, the resulting crude residue was purified by flash chromatography with CH₂Cl₂/MeOH (100:0 to 92:8, v/v) to afford 33 (495 mg, 71%) as a white solid.

TLC: R_{f}=0.33 (CH₂Cl₂/MeOH 92:8);

¹H NMR (400 MHz, CDCl₃): δ=8.47 (s, 1H), 7.43-7.24 (m, 10H), 6.59 (d, J=15.7 Hz, 1H), 5.23 (s, NH₂), 4.92 (s, NH₂), 4.69-4.55 (m, 3H), 4.51 (d, J=11.8 Hz, 1H), 4.37-4.25 (m, 2H), 4.01-3.95 (m, 1H), 3.69-4.62 (m, 1H), 1.15 (d, J=22.4 Hz, 3H);

¹³C NMR (101 MHz, CDCl₃): δ=161.2, 158.8, 148.6, 148.1, 137.3, 128.8, 128.7, 128.4, 128.3, 128.3, 128.2, 105.8, 101.7, 99.9, 90.6, 90.2, 79.8, 77.0, 74.2, 73.7, 67.2, 17.0, 16.8;

¹⁹F NMR (376 MHz, CDCl₃) δ=−163.2;

MS (ESI): m/z 535.19 [M+H]⁺.

### For Compound 34: 2′-deoxy-2′-fluoro-2′-C-methyl-1′-[(5,7-diaminoimidazo-[4′,5′:4,5]thieno[3,2-d]pyrimidin-3-yl)]-β-D-ribofuranose

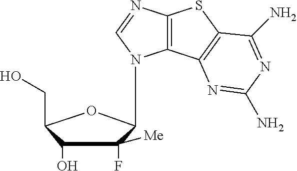

To a solution of 33 (169 mg, 0.316 mmol, 1 eq.) in anhydrous DCM (12 mL), was added BF₃·Et₂O (1.20 mL, 9.48 mmol, 30 eq.) and ethanedithiol (1.30 mL, 15.8 mmol, 50 eq.). The reaction mixture was stirred at room temperature for 48 h. After solvent and excess of reagents removal, the resulting crude residue was purified by flash chromatography on reversed phase with H₂O/MeCN (100:0 to 80:20, v/v) and freeze-dried to afford 34 (85.9 mg, 77%) as a white solid.

¹H NMR (400 MHz, DMSO-d₆): δ=8.49 (s, 1H), 6.84 (s, NH₂), 6.46 (d, J=16.8 Hz, 1H), 5.94 (s, NH₂), 5.68 (d, J=6.9 Hz, 1H), 5.35 (t, J=4.8 Hz, 1H), 4.24 (br d, 1H), 3.94-3.82 (m, 2H), 3.68 (ddd, J=12.4, 4.8, 2.9 Hz, 1H), 1.04 (d, J=22.5 Hz, 3H);

¹³C NMR (101 MHz, DMSO-d₆): δ=161.7, 159.0, 147.3, 147.0, 142.3, 127.1, 103.3, 102.1, 100.3, 89.8, 89.6, 82.0, 70.0, 69.8, 59.0, 16.4, 16.1;

¹⁹F NMR (376 MHz, DMSO-d₆) δ=−163.2;

HRMS-ESI (m/z): calcd for C₁₃H₁₆FN₆O₃S [M+H]⁺ 355.09831. found 355.09863.

HPLC: Method A: H₂O/CH₃CN 95:5 to 20:80 in 8 min, λ=254 nm, t_{R}=3.261 min.

### Example 11

### Synthesis of the Nucleoside Prodrugs (35-37):

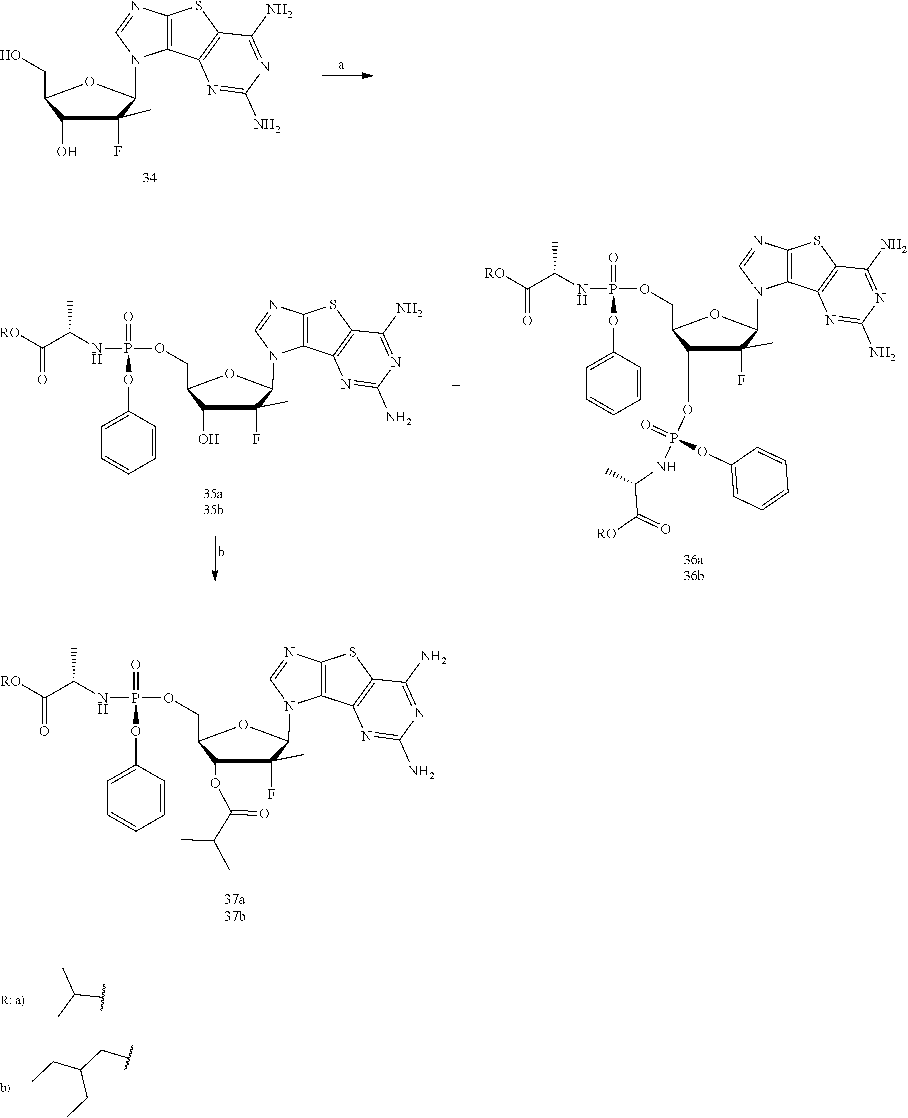

Reagents and conditions: (a) t-BuMgCl, isopropyl ((S)-(perfluorophenoxy)(phenoxy)phosphoryl)-L-alaninate (for McG₁=35a and 36a) or 2-ethylbutyl ((S)-(perfluorophenoxy)(phenoxy)phosphoryl)-L-alaninate (for McG₂=35b and 36b) (1 eq. or 2 eq.), DMF, 5 min MW, 55° C., 65% for 35a, 25% for 36a, 47% for 35b and 16% for 36b; (b), Isobutyric anhydride (1.2 eq.), DMAP (cat), Py. r.t., 3 h for 37a (86%) 2 h for 37b (88%).

### General Procedure A for the Synthesis of ProTides 35-36:

Reaction under microwave irradiation (MWI): To a solution of Nucleoside 34 (20-40 mg, 1 eq.) in anhydrous DMF (0.04 M) in a 10 mL sealed microwave tube under an argon atmosphere, was added a solution of tert-butylmagnesium chloride (1 eq. to 1.5 eq., 1 M solution in THF) at room temperature. The resulting mixture was stirred for 15 min. at room temperature. To this mixture was added the corresponding phosphoramidating reagent (1.2 eq. to 1.5 eq., 0.7 M solution in DMF) dropwise. The microwave vial was capped and placed into the microwave cavity in closed vessel mode and stirred under for 5 min at 55° C. (dynamic method). After cooling down, methanol was added, and solvents removed under reduced pressure. The resulting crude residue was then purified by flash chromatography on reversed phase with H₂O/MeCN (100:0 to 20:80, v/v) and freeze-dryed to afford a mixture of 5′-ProTide and 3′-5′-ProTide as a white solid.

### Synthesis of Compound 5′-ProTide 35a (5′-O[phenyl-(isopropoxy-L-alaninyl)]phosphate, CEM-041) and 3′,5′-ProTide 36a (3′,5′-O[phenyl-(isopropoxy-L-alaninyl)]phosphate; CEM-042)

Compounds 35a and 36a were prepared following the general procedure A, using the nucleoside 34 (30.0 mg, 0.085 mmol), tert-butylmagnesium chloride (0.085 mmol, 1. eq.) and the commercially available isopropyl((S)-(perfluorophenoxy)(phenoxy)phosphoryl)-L-alaninate (0.102 mmol, 1.2 eq.) as a phosphoramidating reagent. Purification of the crude by flash chromatography on reversed phase with H₂O/MeCN (100:0 to 40:60, v/v) and freeze-drying afforded 35a (34.3 mg, 65%) and 36b (18.7 mg, 25%) as white solids.

### 5′-ProTide 35a (5′-O[phenyl-(isopropoxy-L-alaninyl)]phosphate)

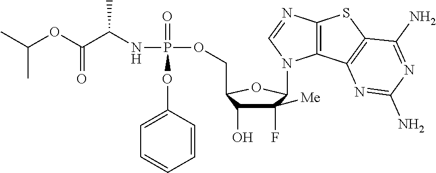

¹H NMR (400 MHz, CD₃OD): δ=8.27 (s, 1H), 7.38-7.22 (m, 4H), 7.17 (t, J=7.2 Hz, 1H), 6.58 (d, J=18.3 Hz, 1H), 4.89-4.80 (m, 1H), 4.64-4.46 (m, 3H), 4.28-4.19 (m, 1H), 3.98-3.86 (m, 1H), 1.31 (d, J=7.1 Hz, 3H), 1.20 (d, J=22.5 Hz, 3H), 1.15 (d, J=6.3 Hz, 3H), 1.12 (d, J=6.3 Hz, 3H);

¹³C NMR (101 MHz, CD₃OD) δ=173.1, 173.0, 161.9, 159.5, 150.8, 150.7, 148.3, 147.4, 142.5, 129.5, 127.2, 124.9, 120.1, 120.1, 104.7, 101.4, 99.6, 91.3, 90.9, 80.0, 79.9, 71.5, 71.3, 68.8, 65.4, 50.3, 48.4, 20.6, 20.5, 19.3, 19.3, 15.5, 15.3;

³¹P NMR (162 MHz, CD₃OD) δ=4.6;

¹⁹F NMR (376 MHz, CD₃OD) δ −163.4;

HRMS-ESI (m/z): calcd for C₂₅H₃₂FN₇O₇PS [M+H]⁺ 624.18001. found 624.18052.

HPLC: Method A: H₂O/CH₃CN 95:5 to 20:80 in 8 min, λ=254 nm, t_{R}=5.538 min.

### 3′,5′-ProTide 36a (3′,5′-O[phenyl-(isopropoxy-L-alaninyl)]phosphate)

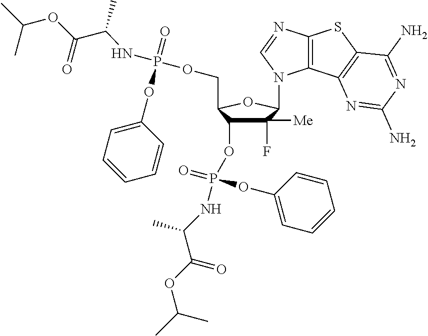

¹H NMR (400 MHz, CD₃OD): δ=8.25 (s, 1H), 7.41-6.80 (m, 11H), 6.27 (d, J=21.9 Hz, 1H), 5.05-4.90 (m, 2H), 4.82-4.71 (m, 2H), 4.70-4.58 (m, 1H), 4.42-4.32 (m, 1H), 3.99 (dq, J=14.3, 7.1 Hz, 1H), 3.91-3.78 (m, 1H), 1.39 (d, J=7.1 Hz, 3H), 1.27-1.15 (m, 12H), 1.08 (d, J=6.3 Hz, 3H), 1.06 (d, J=6.3 Hz, 3H);

¹³C NMR (101 MHz, CD₃OD) δ=173.1, 173.1, 161.6, 159.6, 150.8, 150.7, 150.7, 150.7, 150.0, 147.1, 145.5, 129.5, 129.2, 126.1, 125.1, 124.6, 120.3, 120.3, 120.1, 120.0, 104.2, 100.4, 100.3, 98.5, 98.5, 92.8, 92.2, 78.8, 78.8, 78.7, 78.7, 75.0, 75.0, 74.9, 74.8, 68.8, 68.7, 66.3, 50.5, 50.2, 20.7, 20.6, 20.6, 20.5, 19.2, 19.2, 16.0, 15.8;

³¹P NMR (162 MHz, CD₃OD) δ=5.9, 4.2;

¹⁹F NMR (376 MHz, CD₃OD) δ −155.7;

HRMS-ESI (m/z): calcd for C₃₇H₄₈FN₈O₁₁P₂S [M+H]⁺ 893.26170. found 893.26151.

RP18-HPLC: method A, t_{R}=7.414 min.

### Synthesis of Compound 5′-ProTide 35b (5′-O[phenyl-(2-ethylbutoxy-L-alaninyl)]phosphate, CEM-052) and 3′,5′-ProTide 36b (3′,5′-O[phenyl-(2-ethylbutoxy-L-alaninyl)]phosphate, CEM-053)

Compounds 35b and 36b were prepared following standard procedure A using the nucleoside 34 (50.0 mg, 0.141 mmol), tert-butylmagnesium chloride (0.212 mmol, 1.5 eq.) and the commercially available 2-ethylbutyl ((S)-(perfluorophenoxy)(phenoxy)phosphoryl)-L-alaninate (0.212 mmol, 1.5 eq.) as phosphoramidating reagent. Purification of the crude by flash chromatography on reversed phase with H₂O/MeCN (100:0 to 40:70, v/v) and freeze-drying afforded 35b (44.6 mg, 47%) and 36b (21.8 mg, 16%) as a white solid.

### 5′-ProTide 35b (5′-O[phenyl-(2-ethylbutoxy-L-alaninyl)]phosphate)

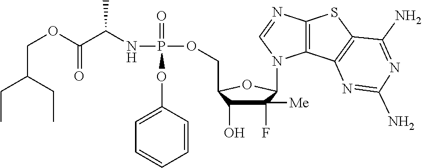

¹H NMR (400 MHz, CD₃OD): δ=8.27 (s, 1H), 7.34 (t, J=7.8 Hz, 2H), 7.26 (d, J=8.4 Hz, 2H), 7.17 (t, J=7.3 Hz, 1H), 6.58 (d, J=18.2 Hz, 1H), 4.63-4.42 (m, 3H), 4.26-4.19 (m, 1H), 4.04-3.91 (m, 2H), 3.83 (dd, J=10.9, 5.7 Hz, 1H), 1.46-1.36 (m, 1H), 1.35-1.23 (m, 7H), 1.20 (d, J=22.5 Hz, 3H), 0.84-0.77 (m, 6H);

¹³C NMR (101 MHz, CD₃OD) δ=175.0, 174.9, 163.2, 160.8, 152.1, 152.1, 149.6, 148.7, 143.8, 130.8, 128.5, 126.2, 121.4, 121.4, 106.0, 102.7, 100.9, 92.6, 92.2, 81.4, 81.3, 72.9, 72.7, 68.1, 66.8, 51.6, 41.6, 24.2, 24.1, 20.7, 20.6, 16.8, 16.6, 11.3, 11.2;

³¹P NMR (162 MHz, CD₃OD) δ 4.5;

¹⁹F NMR (376 MHz, CD₃OD) δ=−163.5;

HRMS-ESI (m/z): calcd for C₂₈H₃₈FN₇O₇PS [M+H]⁺ 666.22696. found 666.22637.

HPLC: Method A: H₂O/CH₃CN 95:5 to 20:80 in 8 min, λ=254 nm, t_{R}=6.559 min.

### 3′,5′-ProTide 36b (3′,5′-O[phenyl-(2-ethylbutoxy-L-alaninyl)]phosphate)

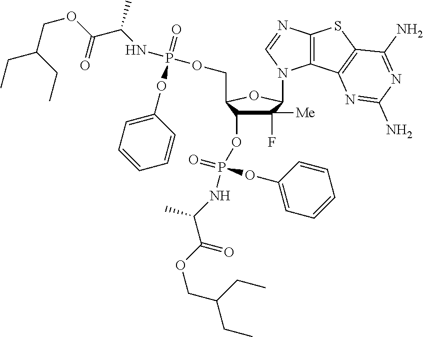

¹H NMR (400 MHz, CD₃OD) δ=8.25 (s, 1H), 7.38-6.71 (m, 11H), 6.27 (d, J=21.8 Hz, 1H), 4.82-4.72 (m, 1H), 4.70-4.58 (m, 1H), 4.42-4.32 (m, 1H), 4.12-3.95 (m, 3H), 3.97-3.84 (m, 2H), 3.86-3.76 (m, 1H), 1.59-1.45 (m, 1H), 1.44-1.22 (m, 15H), 1.19 (d, J=22.8 Hz, 3H), 0.90 (t, J=7.4 Hz, 6H), 0.82 (t, J=7.4 Hz, 6H);

¹³C NMR (101 MHz, CD₃OD) δ=174.9, 174.9, 162.9, 161.0, 152.1, 152.1 152.0, 152.0, 148.4, 130.8, 130.5, 127.5, 126.4, 125.9, 121.6, 121.6, 121.4, 121.3, 105.5, 93.7, 80.0, 76.2, 68.1, 67.9, 67.6, 51.7, 51.5, 41.8, 41.7, 24.3, 24.3, 24.2, 24.2, 20.7, 20.6, 17.3, 17.0, 11.4, 11.4, 11.3, 11.3;

³¹P NMR (162 MHz, CD₃OD) δ=5.8, 4.1;

¹⁹F NMR (376 MHz, CD₃OD) δ=−155.7;

HRMS-ESI (m/z): calcd for C₄₃H₆₀FN₈O₁₁P₂S [M+H]⁺ 977.35560. found 977.35158.

HPLC: Method A: H₂O/CH₃CN 95:5 to 20:80 in 8 min, λ=254 nm, t_{R}=9.186 min.

### Synthesis of the Nucleoside Prodrug 37a (CEM-054):

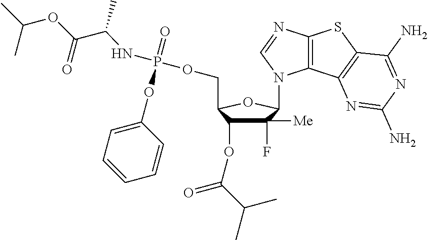

To a solution of 35a (25.3 mg, 0.0406 mmol) in anhydrous pyridine (2.0 mL), was added isobutyric anhydride (8.07 μL, 0.0487 mmol, 1.2 eq.) and a catalytic amount of DMAP. The reaction mixture was stirred at room temperature for 3 h. After solvent removal, the resulting crude residue was purified by flash chromatography with CH₂Cl₂/MeOH (100:0 to 90:10, v/v) to afford 37a (24.2 mg, 86%) as a white solid.

¹H NMR (400 MHz, CD₃OD) δ=8.29 (s, 1H), 7.32-7.22 (m, 2H), 7.22-7.00 (m, 4H), 6.38 (d, J=21.4 Hz, 1H), 4.79 (hept, J=6.4 Hz, 1H), 4.58-4.30 (m, 3H), 3.91-3.77 (m, 1H), 2.73 (hept, J=7.0 Hz, 1H), 1.35-1.18 (m, 12H), 1.11 (dd, J=9.6, 6.3 Hz, 6H);

¹³C NMR (101 MHz, CD₃OD) δ=178.5, 174.3, 174.3, 162.9, 161.0, 152.0, 152.0, 151.0, 148.4, 146.3, 130.6, 127.7, 126.0, 121.4, 121.3, 105.7, 102.2, 100.3, 94.0, 93.6, 79.6, 79.5, 73.3, 73.2, 70.0, 67.3, 51.5, 35.1, 21.9, 21.8, 20.5, 20.4, 19.4, 19.2, 17.6, 17.3;

¹⁹F NMR (376 MHz, CD₃OD) δ=−154.9;

³¹P NMR (162 MHz, CD₃OD) δ=4.3;

HRMS-ESI (m/z): calcd for C₂₉H₃₈FN₇O₈PS [M+H]⁺ 694.22187. found 694.22060.

HPLC: Method A: H₂O/CH₃CN 95:5 to 20:80 in 8 min, λ=254 nm, t_{R}=6.852 min.

### Synthesis of the Nucleoside Prodrug 37b (CEM-055):

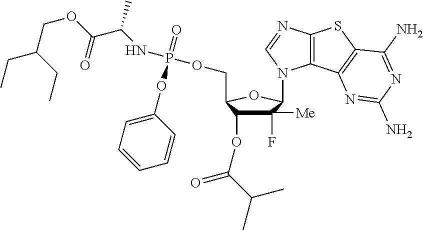

To a solution of 35b (50.0 mg, 0.0751 mmol) in anhydrous pyridine (2.0 mL) was added isobutyric anhydride (14.9 μL, 0.0901 mmol, 1.2 eq.) and a catalytic amount of DMAP. The reaction mixture was stirred at room temperature for 2 h. After solvent removal, the resulting crude residue was purified by flash chromatography with CH₂Cl₂/MeOH (100:0 to 90:10, v/v) to afford 37b (48.5 mg, 88%) as a white solid.

TLC: R_{f}=0.34 (CH₂Cl₂/MeOH 90:10);

¹H NMR (400 MHz, CD₃OD) δ=8.29 (s, 1H), 7.32-7.22 (m, 2H), 7.20-6.97 (m, 4H), 6.38 (d, J=21.3 Hz, 1H), 4.57-4.47 (m, 1H), 4.46-4.40 (m, 1H), 4.39-4.30 (m, 1H), 3.99-3.79 (m, 3H), 2.81-2.64 (m, 1H), 1.47-1.32 (m, 1H), 1.33-1.18 (m, 16H), 0.87-0.77 (m, 6H);

¹³C NMR (101 MHz, CD₃OD) δ=178.4, 174.9, 174.8, 162.9, 161.0, 152.0, 152.0, 151.0, 148.5, 146.2, 130.7, 127.7, 126.1, 121.4, 121.3, 105.8, 102.2, 100.3, 94.0, 93.6, 79.6, 79.6, 73.4, 73.2, 67.9, 67.3, 51.5, 41.7, 35.1, 24.2, 20.6, 20.5, 19.4, 19.2, 17.6, 17.3, 11.3;

³¹P NMR (162 MHz, CD₃OD) δ=4.2;

¹⁹F NMR (376 MHz, METHANOL-D₃) δ=−155.1;

HRMS-ESI (m/z): calcd for C₂₉H₃₈FN₇O₈PS [M+H]⁺ 694.22187. found 694.22060.

HPLC: Method A: H₂O/CH₃CN 95:5 to 20:80 in 8 min, λ=254 nm, t_{R}=7.758 min.

### Example 12

### Synthesis of Nucleoside 5′-Triphosphates (38-39):

### General Procedure B: Synthesis of Nucleoside 5′-Triphosphates:

In a Schlenk flask, the starting material (20 or 30 mg 1 eq.) was suspended with anhydrous pyridine (150 μL) and put under a high vacuum overnight. The resulting dry residue was then dissolved with dry trimethyl phosphate (0.1 mL/mg) under argon and cooled down to 0° C., tributylamine (3 eq.) and POCl₃ (15 eq.) were then added. The reaction mixture was then stirred at 0° C. to convert the starting material to the corresponding monophosphate. After 85 to 100% of conversion (4-16 h) is observed by HPLC, a solution of tetrabutylammonium salt of pyrophosphate (15 eq.) and tributylamine (6 eq.) in MeCN (0.5-1 mL) was added at 0° C. The reaction was monitored by HPLC and allowed to stir at 0° C. until 85 to 100% of the monophosphate is converted to the corresponding nucleoside triphosphate. The reaction was stopped by adding an aqueous triethylammonium bicarbonate solution (TEAB, 1M, pH 7) and concentrated to dryness under reduced pressure. The resulting crude residue was purified by flash chromatography on a Sephadex column (20 mL/min) with H₂O/TEAB (IM) (100:0 to 0:100, v/v) to afford the desired compound along with an excess of triethylammonium salts. A second purification by flash chromatography on reverse phase (C18Aq) with H₂O/MeCN (100:0 to 0:100, v/v) was then performed and afforded the title compound as tris triethylammonium salts after freeze-drying.

### Synthesis of the Trisphosphate 38 (CEM-038):

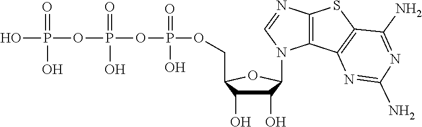

The title compound was obtained as a hygroscopic solid (13.2 mg, 16%) from compound 21 (30 mg, 0.088 mmol) following general procedure B.

¹H NMR (400 MHz, D₂O) δ=8.33 (s, 1H), 6.09 (d, J=5.4 Hz, 1H), 4.71 (t, J=5.3 Hz, 1H), 4.53-4.45 (m, 1H), 4.47-4.40 (m, 1H), 4.30 (ddd, J=11.2, 5.2, 3.6 Hz, 2H), 4.19 (ddd, J=11.2, 6.8, 4.1 Hz, 2H), 3.12 (q, J=7.3 Hz, 18H), 1.21 (t, J=7.3 Hz, 29H);

³¹P NMR (162 MHz, D₂O) δ=−9.87 (d, J=19.6 Hz, P-α), −10.86 (d, J=19.8 Hz, P-γ), −22.59 (t, J=19.7 Hz, P-β);

HRMS-ESI (m/z): calcd for C₁₂H₁₆N₆O₁₃P₃S [M−H]⁻ 576.97144. found 576.97021.

HPLC: Method B: TBAA-Buffer/CH₃CN 95:5 to 20:80 in 8 min, λ=254 nm, t_{R}=5.164 min.

### Synthesis of the Trisphosphate 39 (CEM-051):

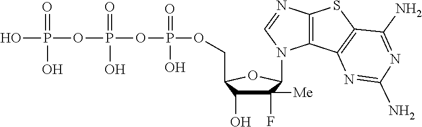

The title compound was obtained as a hygroscopic solid (12.7 mg, 18%) from compound 34 (30 mg, 0.085 mmol) following general procedure B.

¹H NMR (400 MHz, D₂O) δ=8.30 (s, 1H), 6.34 (d, J=16.3 Hz, 1H), 4.58-4.50 (m, 1H), 4.35-4.20 (m, 3H), 3.12 (q, J=7.3 Hz, 15H), 1.20 (t, J=7.3 Hz, 22.5H), 0.97 (d, J=23.2 Hz, 3H);

³¹P NMR (162 MHz, D₂O) δ=−9.12 (d, J=19.6 Hz, P-α), −10.76 (d, J=19.3 Hz, P-γ), −22.27 (t, J=19.4 Hz, P-β);

HRMS-ESI (m/z): calcd for C₁₃H₁₇FN₆O₁₂P₃S [M−H]⁻ 592.98275. found 592.98159.

HPLC: Method B: TBAA-Buffer/CH₃CN 95:5 to 20:80 in 8 min, λ=254 nm, t_{R}=5.321 min.

### Example 13

The results of the effectiveness of specific structures relating to different viruses are shown below. When reviewing the results, it should be noted that the lower the IC₅₀, the lower the concentration of drug required to produce 50% of maximum effect and thus, the higher the potency of the drug.

TABLE 1
IC₅₀ results.

| Virus | CEM-007 | CEM-010 | CEM-012 | CEM-015 |
|---|---|---|---|---|
| DENV2 | IC50 = 45 μM | | | |
| DENV4 | IC50 = 64 μM | | | |
| SARS-CoV-2 (Omicron) | IC50 = 56.4 μM | | | |
| (Calu3) #1 | | | | |
| EBOV (VP30, Huh) | IC50 = 15.2 & | | IC50 = 49.3 μM | |
| | 11.7 μM (Huh7) | | CC50 = 17.7 μM | |
| | EC50-40.5 μM | | | |
| | CC50 >100 μM | | | |
| CHIKV | | IC50 = 82.46 μM | | IC50 = 93.91 μM |
| | | CC50 >100 μM | | Non-tox at 100 μM |

| Virus | CEM-021 | CEM-024 | CEM-028 | CEM-036 |
|---|---|---|---|---|
| DENV2 | IC50 = 73.5 μM | | | IC50 = 24.0 μM |
| | CC50 >100 μM | | | CC50 >100 μM |
| DENV4 | IC50 = 100 μM | | | IC50 = 34.0 μM |
| SARS-CoV-2 (Omicron) | | | | IC50 = >100 μM |
| (Calu3) #1 | | | | CC50 >100 μM |
| SARS-CoV-2 (WA-1) | | | | IC50 = 21.63 μM |
| #1 | | | | CC50 >100 μM |
| SARS-CoV-2 (WA-1) | | | | IC50 = 48.35 μM |
| #2 | | | | CC50 >100 μM |
| EBOV (VP30, Huh) | IC50 = 43.6 μM | IC50 = 31 μM | IC50 = 48 μM | IC50 = 37.7 μM |
| | CC50 = 60.5 μM | (Huh7) | (Huh7) | (Huh7) |
| | | CC50 = 11 μM | CC50 = 138 μM | CC50 = >200 μM |

| Virus | CEM-041 | CEM-042 | CEM-052 | CEM-053 |
|---|---|---|---|---|
| DENV2 | | IC50 = 3.5 μM | IC50 = 19 μM | IC50 = 18 μM |
| | | IC90 = 6.8 μM | | |
| | | CC50 >100 μM | | |
| DENV4 | | IC50 = 7.5 μM | IC50 = 16 μM | IC50 = 2.9 μM |
| | | IC90 = 25 μM | CC50 = 33 μM | CC50 >50 μM |
| | | CC50 >100 μM | | |
| SARS-CoV-2 (Omicron) | IC50 = >100 μM | IC50 = 7.36 μM | IC50 = 20.557 μM | IC50 = 21.866 μM |
| (Calu3) #1 | CC50 >100 μM | CC50 >100 μM | CC50 = 44.34 μM | CC50 >100 μM |
| SARS-CoV-2 (WA-1) | IC50 = 73.82 μM | IC50 = 3.58 μM | | |
| #1 | CC50 >100 μM | CC50 >100 μM | | |
| SARS-CoV-2 (WA-1) | | IC50 = 5.89 μM | | IC50 = 2.947 μM |
| #2 | | CC50 >100 μM | | |
| SARS-CoV-2 (A549- | | EC50 = 5.89 μM | | |
| hACE2) | | EC90 = 6.5 μM | | |
| | | CC50 >100 μM | | |
| EBOV (VP30, Huh) | | IC50 = 11.6 μM | EC50 = 9.1 μM | EC50 = 4.5 μM |
| | | (Huh7) | EC50 = 11.4 μM | EC50 = 6.7 μM |
| | | CC50 = 49 μM | CC50 = 88.5 μM | CC50 = 73.7 μM |
| CHIKV | | EC50 = 24.62 μM | | |
| | | CC50 >100 μM | | |
| VEEV | | EC50 = 86.7 μM | | |
| | | CC50 >100 μM | | |
| OC43 | | EC50 = 1.50 μM | | |
| | | (Huh 7.5 | | |
| | | IFITM3) | | |

| Virus | CEM-054 | CEM-055 | CEM-056 |
|---|---|---|---|
| DENV2 | IC50 = 22 μM | | |
| DENV4 | IC50 = 10 μM | | |
| | CC50 = 32 μM | | |
| SARS-CoV-2 (Omicron) | IC50 = 37.932 μM | IC50 = 19.644 μM | IC50 = 59.252 μM |
| (Calu3) #1 | CC50 >100 μM | CC50 = 55.322 μM | CC50 >100 μM |
| SARS-CoV-2 (WA-1) | | | |
| #1 | | | |
| SARS-CoV-2 (WA-1) | | IC50 = 11.89 μM | IC50 = 44.72 μM |
| #2 | | | |
| EBOV (VP30, Huh) | EC50 = 10.8 μM | EC50 = 6.2 μM | EC50 = 22.1 μM |
| | EC50 = 11.2 μM | EC50 = 10.1 μM | CC50 >50 μM |
| | CC50 = 68.8 μM | CC50 = 95.8 μM | |

DENV2 = Dengue Virus 2; DENV4 = Dengue Virus 4; EBOV = Ebola Virus; CHIKV = Chikungunya Virus; VEEV = Venezuelan Equine Encephalitis Virus; OC43 = human coronavirus; SARS-CoV-2 (Omicron) was the Omicron strain in Calu-3 cells; SARS-CoV-2 (WA-1) was the WA-1 strain in A549 cells; #1 was a different lab than #2.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

[1]K. L. Seley, et al. J. Org. Chem. 2002, 67, 3365-3373.
[2]K. L. Seley, et al. J. Org. Chem. 2005, 70, 1612-1619.
[3]K. L. Seley, et al. Org. Lett. 2005, 7(1), 63-66.
[4]Z. Chen, et al. ACS Infect. Dis. 2015, 1, 357-366.

That which is claimed is:

1. A nucleoside compound selected from at least one of formula (I) or formula (II): 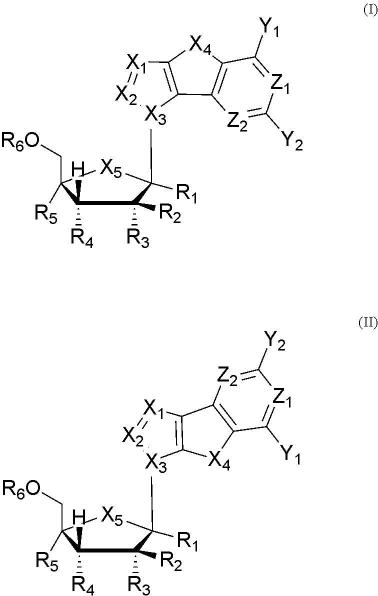
wherein
X₁ and X₂ are each independently selected from C, O, N, or S;
X₃ is C or N;
X₄ is O, NH, S, or CH₂;
X₅ is O, NH, S, or CH₂;
Y₁ and Y₂ are each independently selected from H, OH, SH, ═O, F, NH₂, NH-alkyl, O-alkyl, NH—OH, alkyl, CF₃, Cl, CN, or N₃;
Z₁ and Z₂ are each independently selected from C or a heteroatom;
R₁ is H or cyano;
R₂ and R₃ are each independently selected from H, OH, F, Cl, alkyl, ethynyl, or various prodrugs (e.g., amino acid prodrugs);
R₄ is H, OH, N₃, or various prodrugs (e.g., amino acid prodrugs);
R₅ is H, F, ethynyl or cyano; and
R₆ is H, monophosphate, diphosphate, triphosphate, or various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof;
and/or formula (III) 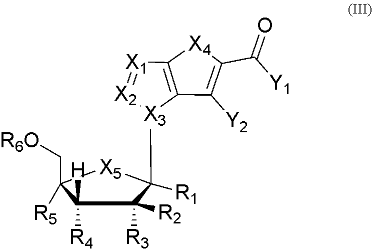
wherein
X₁ and X₂ are each independently selected from C, O, N, or S;
X₃ is C or N;
X₄ is O, NH, S, or CH₂;
X₅ is O, NH, S, or CH₂;
Y₁ is OH, NH₂, NH-alkyl, O-alkyl or NH—Ar
Y₂ is NH₂, NH-alkyl, O-alkyl or NH—Ar
R₁ is H or cyano;
R₂ and R₃ are each independently selected from H, OH, F, Cl, alkyl, ethynyl, or various prodrugs (e.g., amino acid prodrugs);
R₄ is H, OH, N₃, or various prodrugs (e.g., amino acid prodrugs);
R₅ is H, F, ethynyl or cyano; and
R₆ is H, monophosphate, diphosphate, triphosphate, or various prodrugs (e.g., esters, McGuigan ProTides, lipid phosphates, lipid esters, HepDirect, amino acid prodrugs), or a pharmaceutically acceptable salt, isomer, hydrate, prodrug or solvate thereof,
or a pharmaceutically acceptable salt, isomer, hydrate, prodrug or solvate thereof.

2. The nucleoside compound of claim 1, wherein X₁═X₃═N and X₂ is C.

3. The nucleoside compound of claim 1, wherein Z₁═Z₂═N.

4. The nucleoside compound of claim 1, wherein X₄ is S.

5. The nucleoside compound of claim 1, wherein R₁═R₅═H.

6. The nucleoside compound of claim 1, wherein R₂ and R₃ are the same as or different from one another and are selected from H, OH, F, or methyl.

7. The nucleoside compound of claim 1, wherein R₄ is OH or a prodrug.

8. The nucleoside compound of claim 1, wherein R₆ is one of H, McGuigan ProTide, or triphosphate.

9. The nucleoside compound of claim 1, wherein the nucleoside compound is selected from (i) formula (I) or (II), wherein Y₁ and Y₂ are the same as or different from one another and are selected from H, OMe, NH₂, or ═O; or (ii) formula (III), wherein Y₁ and Y₂ are the same as or different from one another and are selected from H, OMe, or NH₂.

10. A nucleoside compound selected from the group consisting of:
CEM-001: 1-[5-(Pyrimidin-6-yl-4-one)imidazol-1-yl]-1-β-D-ribofuranose 2,3,5-Triol (compound 11) 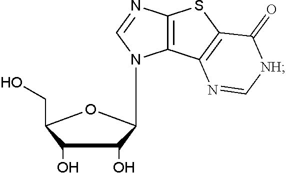
CEM-002: 1-[(5-Hydroxylimidazo[4′,5′:4,5]thieno[3,2-d]pyrimidin-3-yl-7-one)]-β-D-ribofuranose 2,3,5-Triol (compound 16) 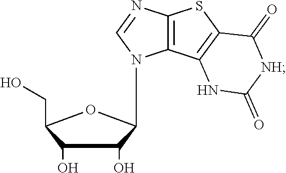
CEM-004: 1-[(5-carboxamide)-thieno[2,3-d]imidazol-3-yl]-β-D-ribofuranose 2,3,5-Triol (compound 6) 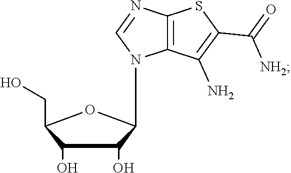
CEM-007: 1-[(5,7-diaminoimidazo-[4′,5′:4,5]thieno[3,2-d]pyrimidin-3-yl)]-β-D-ribofuranose-2,3,5-Triol (compound 21) 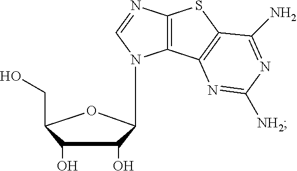
CEM-009: Nucleoside prodrug (compound 19) 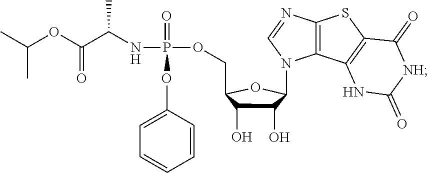
CEM-010: Nucleoside prodrug (compound 14) 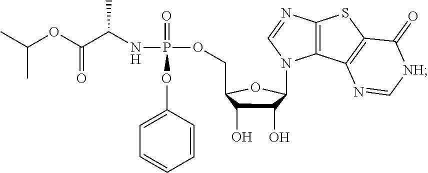
CEM-011: Nucleoside prodrug (compound 24a) 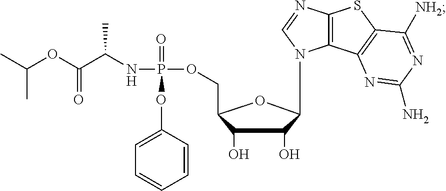
CEM-012: Nucleoside prodrug (compound 29) 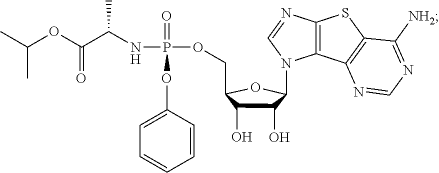
CEM-015: Nucleoside prodrug (compound 9) 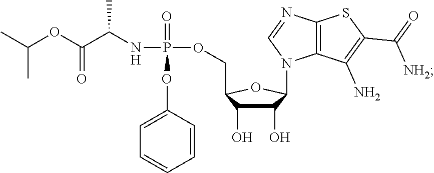
CEM-021: 1-[(5-Aminoimidazo[4′,5′:4,5]thieno[3,2-d]pyrimidin-3-yl-7-one)]-β-D-ribofuranose 2,3,5-Triol 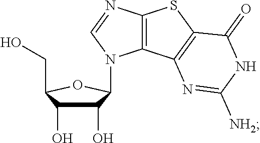
CEM-024: 2′-deoxy-2′-fluoro-2′-C-methyl-1′-[(5,7-diaminoimidazo-[4′,5′:4,5]thieno[3,2-d]pyrimidin-3-yl)]-β-D-ribofuranose (compound 34) 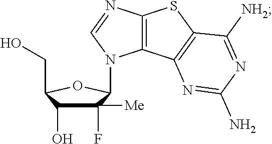
CEM-028: 1-[(5,7-dimethoxyimidazo-[4′,5′:4,5]thieno[3,2-d]pyrimidin-3-yl)]-β-D-ribofuranose-2,3,5-Triol 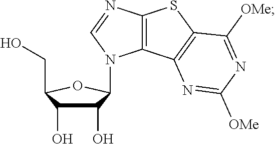
CEM-033: Nucleoside prodrug 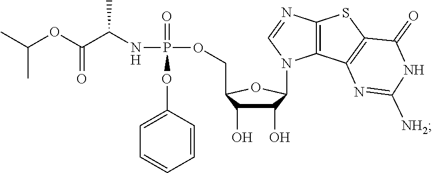
CEM-036: Nucleoside prodrug 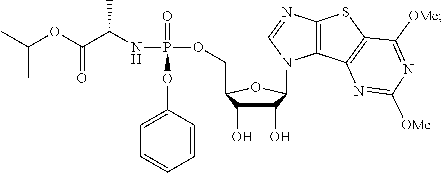
CEM-038: Trisphosphate compound (compound 38) 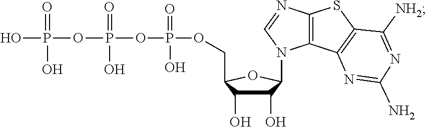
CEM-041: Nucleoside prodrug (compound 35a) 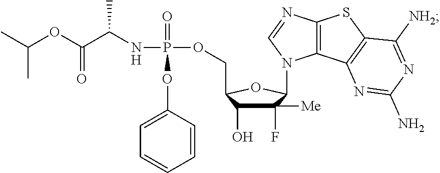
CEM-042: Nucleoside prodrug (compound 36a) 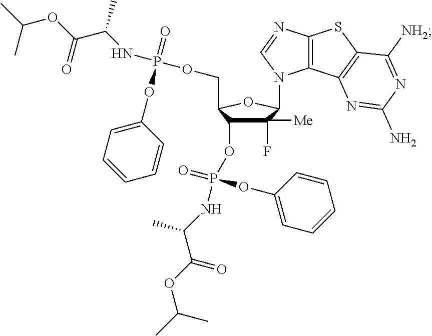
CEM-051: Trisphosphate compound (compound 39) 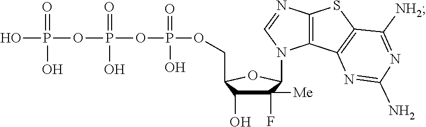
CEM-052: Nucleoside prodrug (compound 35b) 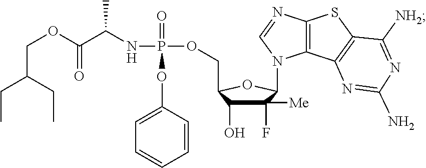
CEM-053: Nucleoside prodrug (compound 36b) 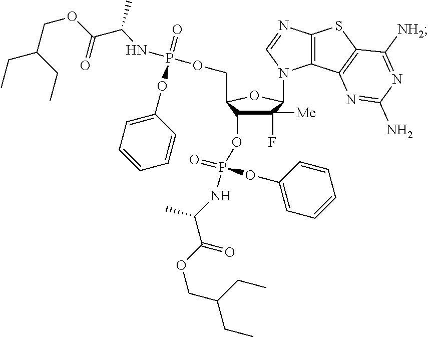
CEM-054: Nucleoside prodrug (compound 37a) 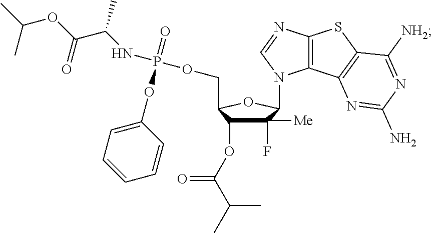
CEM-055: Nucleoside prodrug (compound 37b) 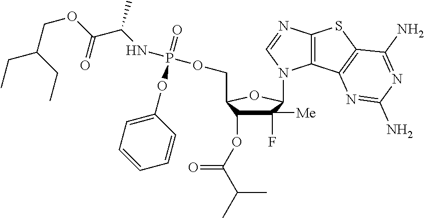
CEM-056: Nucleoside prodrug (compound 24b) 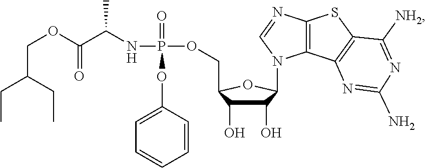
or a pharmaceutically acceptable salt, isomer, hydrate, prodrug or solvate thereof.

11. A pharmaceutical composition comprising at least one of the nucleoside compounds of claim 1 and at least one pharmaceutically acceptable carrier.

12. The pharmaceutical composition of claim 11, further comprising at least one additional pharmaceutically active agent.

13. The pharmaceutical composition of claim 11, formulated as a syrup, elixir, tablet, troche, lozenge, hard or soft capsule, pill, suppository, oily or aqueous suspension, dispersible powder or granule, emulsion, injectable, solution, sustained release formulation, or aerosol.

14. A method for treating and/or preventing a viral infection in a subject, wherein the viral infection is caused by at least one of a coronavirus, a herpesvirus, an alphavirus, a polyomavirus, an enterovirus, a filovirus, a matonavirus, a phenuivirus, Hepatitis B virus, and/or a flavivirus, comprising administration, to the subject, of a therapeutically effective amount of at least one nucleoside compound of claim 1.

15. The method of claim 14, wherein a therapeutically effective amount of the nucleoside compound is from 0.05 to 50 mg per kilogram body weight of the subject per day.

16. The method of claim 14, wherein the method of administration is selected from the group consisting of systemically, orally, buccally, sublingually, topically, by inhalation, by spraying, intravenously, intramuscularly, subcutaneously, intrathecally, intradermally, intravascularly or intra-arterially.

17. The method of claim 14, wherein the viral infection is caused by:
a coronavirus selected from human coronaviruses (HCoV), Severe Acute Respiratory Syndrome Coronavirus (SARS-CoV), SARS-CoV-2, and Middle East respiratory syndrome (MERS), and mutants thereof;
a herpesvirus selected from herpes simplex virus 1, herpes simplex virus 2, varicella-zoster virus, Epstein-Barr virus, cytomegalovirus, Human herpesvirus-6, Human herpesvirus-7, and Kaposi's sarcoma herpes virus;
an alphavirus selected from eastern equine encephalomyelitis (EEE), Venezuelan equine encephalomyelitis (VEE), western equine encephalomyelitis (WEE), and Chikungunya;
a polyomavirus;
an enterovirus selected from echovirus and coxsackievirus;
a filovirus selected from Zaire Ebola virus, Sudan Ebola virus, Reston Ebola virus, Cote d'Ivoire Ebola virus and Marburg virus;
a matonavirus selected from Rubella, Rustrela, and Ruhugu;
a phenuivirus selected from Rift Valley Fever virus; and/or
a flavivirus selected from the group consisting of yellow fever virus, Apoi virus, Aroa virus, Bagaza virus, Banzi virus, Bouboui virus, Bukalasa bat virus, Cacipacore virus, Carey Island virus, Cowbone Ridge virus, Dakar bat virus, dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Phenh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, tick-borne encephalitis virus, Tyuleniy virus, Uganda S virus, Usutu virus, Wesselsbron virus, West Nile virus, Yaounde virus, Yokose virus, Zika virus, cell fusing agent virus and Tamana bat virus.

18. The method of claim 14, wherein the viral infection is caused by a Hepatitis B virus.
